(19) United States
(12) Patent Application Publication
(10) Pub. No.: US 2023/0355670 A1
(43) Pub. Date: Nov. 9, 2023

(54) Methods of activating cytotoxic leukocytes using PTP1B and PTPN2 inhibitors

(71) Applicants: Monash University, Clayton, Victoria (AU); Peter MacCallum Cancer Institute, Melbourne, Victoria (AU)

(72) Inventors: Tony TIGANIS, Clayton, Victoria (AU); Florian WIEDE, Melbourne, Victoria (AU)

(21) Appl. No.: 17/780,658

(22) PCT Filed: Dec. 4, 2020
(86) PCT No.: PCT/AU2020/051328

§ 371 Date: May 27, 2022

(30) Foreign Application Priority Data
Dec. 4, 2019 (AU) 2019904589

(51) Int. Cl. A61K 35/17; C12N 5/0783; C12N 15/113; A61P 35/00

(52) U.S. Cl. CPC A61K 35/17; C12N 5/0638; C12N 15/1137; A61P 35/00; C12Y 301/03048

(57) ABSTRACT

The present invention generally relates to methods of activating cells via the inhibition of PTP1B and PTPN2 for use in therapy. For example, the invention relates to preparing cells ex vivo for use in immunotherapy, particularly cancer immunotherapy. More specifically, the invention relates to methods for the preparation of leukocytes, particularly T cells, exhibiting cytotoxic properties for use in adoptive cell transfer. The invention also relates to cells and compositions including them for cancer immunotherapy. The invention also relates to methods of immunotherapy, particularly cancer immunotherapy.

### FIELD OF THE INVENTION

The present invention generally relates to methods of activating cells for use in therapy. For example, the invention relates to preparing cells ex vivo for use in immunotherapy, particularly cancer immunotherapy. More specifically, the invention relates to methods for the preparation of leukocytes, particularly T cells, exhibiting cytotoxic properties for use in adoptive cell transfer. The invention also relates to cells and compositions including same for use in cancer immunotherapy. The invention also relates to methods of immunotherapy, particularly cancer immunotherapy.

### RELATED APPLICATION

This application claims priority from Australian provisional application AU 2019904589, the contents of which are hereby incorporated by reference in their entirety.

### BACKGROUND OF THE INVENTION

Immunotherapy is the use of the immune system of a patient to reject a disease, such as cancer or viral infection, by stimulating the patient's immune system to attack the malignant tumour or virally infected cells (and spare the normal cells of the patient). One mode of immunotherapy employs immunization of the patient (e.g., by administering a cancer vaccine) to train the patient's immune system to recognize and destroy tumour cells. Another approach uses the administration of therapeutic antibodies, thereby recruiting the patient's immune system to destroy tumour cells. Cell-based immunotherapy is another approach, which involves immune cells such as the Natural killer Cells (NK cells), Lymphokine Activated killer cell (LAK), Cytotoxic T Lymphocytes (CTLs), Dendritic Cells (DC), etc.

Many kinds of tumour cells or viral infected cells are tolerated by the patient's own immune system, as they are the patient's own cells (e.g., they are self) and are not effectively recognised by the patient's immune system allowing the tumour or viral infected cells to grow and divide without proper regulatory control. In addition, tumour-specific T cells are normally tolerised so that they do not respond to tumour activity. Accordingly, the patient's own immune system requires stimulation to attack the diseased cells.

Adoptive cell transfer (ACT) is an effective form of immunotherapy and involves the transfer of immune cells with anti-tumour or anti-viral activity into patients. ACT is a treatment approach that typically involves the identification of lymphocytes with anti-tumour or anti-viral activity, the in vitro expansion of these cells to large numbers and their infusion into the disease bearing host.

Adoptive T cell therapy depends on the ability to optimally select or genetically engineer cells with targeted antigen specificity and then induce the T cells to proliferate while preserving their effector function and engraftment and homing abilities. However, clinical trials have been carried out with adoptively transferred cells that were cultured in what are now understood to be suboptimal conditions that impair the essential functions of T cells such as antigen specific cytotoxic activity.

The methods which are currently used to prepare cells for use in adoptive cell therapy are limited in that they provide cells that have less than the expected cell killing of target cells, such as tumour cells. There is therefore a need for new or improved methods and/or compositions for adoptive cell therapy or for preparing cells for use in adoptive cell therapy.

There is also a separate need for new or improved methods and/or compositions for stimulating the immune system for the treatment of cancer.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention relates to a method for producing a leukocyte that has an enhanced capacity for killing a target cell, the method comprising
contacting the leukocyte with a PTP1B inhibitor and a PTPN2 inhibitor in conditions for enabling the inactivation of PTP1B and PTPN2 in the leukocyte,
thereby producing a leukocyte that has an enhanced capacity for killing a target cell.

The present invention relates to a method for producing a leukocyte that has an enhanced capacity for killing a target cell, the method comprising
contacting the leukocyte ex vivo with a PTP1B inhibitor and a PTPN2 inhibitor for a sufficient time and under conditions for inactivation of PTP1B and PTPN2 in the leukocyte,
thereby producing a leukocyte cell that has an enhanced capacity for killing a target cell.

The present invention relates to a method for preparing an ex vivo population of leukocytes exhibiting at least one property of a cytotoxic leukocyte comprising culturing leukocytes in the presence of a PTP1B inhibitor and a PTPN2 inhibitor. Preferably, the method comprises expanding the cells in culture.

In one embodiment, the present invention also provides a method for preparing an ex vivo population of T cells exhibiting at least one property of a cytotoxic T cell comprising the steps of:
culturing a T cell population from a biological sample in the presence of a PTP1B inhibitor and a PTPN2 inhibitor;
expanding the cells in culture;
thereby preparing an ex vivo population of T cells exhibiting cytotoxic properties. Preferably the biological sample is derived from a subject having a cancer or have been conditioned or engineered to have specificity for a cancer.

The present invention relates to an ex vivo method for preparing a composition comprising antigen-specific cytotoxic leukocytes, the method comprising:
providing a biological sample containing a population of leukocytes;
co-culturing antigenic material with the leukocyte population in the presence of a PTP1B inhibitor and a PTPN2 inhibitor; and
expanding the cells in culture,
thereby preparing a composition comprising antigen-specific cytotoxic leukocytes ex vivo.

The present invention relates to a method for expanding a population of leukocytes, the method comprising
contacting a population of leukocytes with a PTP1B inhibitor and a PTPN2 inhibitor in conditions for enabling inactivation of PTP1B and PTPN2 in the leukocytes,
thereby expanding the population of leukocytes.

In any embodiment of the present invention, the leukocytes may comprise T cells, or Natural Killer (NK) cells. Preferably, the leukocytes comprise T cells including CD4+ and CD8+ T cells. The T cells may also include effector and effector memory T cells and/or central memory T cells. The leukocytes (preferably T cells or NK cells) may also be genetically engineered to express anti-tumour T cell receptors or chimeric antigen receptors (CARs), or may be γδ (gamma/delta) T cells. The leukocytes may also comprise tumour infiltrating lymphocytes, peripheral blood lymphocyte, or be enriched with mixed lymphocyte tumour cell cultures (MLTCs) or cloned using autologous antigen presenting cells and tumour derived peptides. The leukocytes may be isolated from a histocompatible donor, or from a cancer-bearing subject. The leukocytes may be obtained from differentiating isolated ESCs or iPSCs obtained from a donor, or from the subject requiring treatment.

The present invention also provides a method for proliferating, enriching or expanding a composition of cells comprising a cytotoxic leukocyte, preferably a CD8+ T cell, that has been modified so that PTPN2 is partially, substantially or completely inhibited in the cell, the method comprising culturing a composition of leukocytes, preferably T cells, in a medium, the medium comprising a PTP1B inhibitor, wherein the PTP1B inhibitor is provided in the medium to permit contact with a cytotoxic leukocyte, preferably CD8+ T cell during culture. Preferably the proliferating, enriching or expanding will result in a doubling of the number of cytotoxic leukocytes, preferably CD8+ T cells that exhibit at least one cytotoxic property. More preferably the cell expansion result in 3× or 4× number of cytotoxic leukocytes, preferably CD8+ T cells that exhibit at least one cytotoxic property. The expansion of cytotoxic leukocytes may be 5×, 6×, 7×, 8×, 9× or over 10×. The method may also increase the relative number of cytotoxic leukocytes, preferably CD8+ T cells in the composition that exhibit at least one cytotoxic property.

The present invention also provides a method for proliferating, enriching or expanding a composition of cells comprising a cytotoxic leukocyte, preferably a CD8+ T cell, that has been modified so that PTP1B is partially, substantially or completely inhibited in the cell, the method comprising culturing a composition of leukocytes, preferably T cells, in a medium, the medium comprising a PTPN2 inhibitor, wherein the PTPN2 inhibitor is provided in the medium to permit contact with a cytotoxic leukocyte, preferably a CD8+ T cell during culture. Preferably the proliferating, enriching or expanding will result in a doubling of the number of cytotoxic leukocytes, preferably CD8+ T cells that exhibit at least one cytotoxic property. More preferably the cell expansion result in 3× or 4× number of cytotoxic leukocyte, preferably CD8+ T cells that exhibit at least one cytotoxic property. The expansion of CD8+ T cells may be 5×, 6×, 7×, 8×, 9× or over 10×. The method may also increase the relative number of cytotoxic leukocytes, preferably CD8+ T cells in the composition that exhibit at least one cytotoxic property.

The present invention also provides a method for proliferating, enriching or expanding a composition of cells comprising a cytotoxic leukocyte, preferably a CD8+ T cell, the method comprising culturing a composition of leukocytes in a medium, the medium comprising a PTP1B inhibitor and a PTPN2 inhibitor, wherein the PTP1B inhibitor and PTPN2 are provided in the medium to permit contact with a cytotoxic leukocyte, preferably a CD8+ T cell during culture. Preferably the proliferating, enriching or expanding will result in a doubling of the number of cytotoxic leukocytes, preferably CD8+ T cells that exhibit at least one cytotoxic property. More preferably the cell expansion result in 3× or 4× number of cytotoxic leukocytes, preferably CD8+ T cells that exhibit at least one cytotoxic property. The expansion of cytotoxic leukocytes, preferably CD8+ T cells may be 5×, 6×, 7×, 8×, 9× or over 10×. The method may also increase the relative number of cytotoxic leukocytes, preferably CD8+ T cells in the composition that exhibit at least one cytotoxic property.

In any embodiment of the above methods, the cytotoxic leukocyte, preferably a CD8+ T cell, has been genetically modified so that PTP1B and/or PTPN2 is partially, substantially or completely inhibited in the cells. In one embodiment, the cell may have been subjected to CRISPR-cas9-RNP to partially or completely ablate expression of the PTPN1 and/or PTPN2 genes although it will be appreciated that any method for genetic modification may be used.

The present invention also relates to a composition of cytotoxic cells wherein greater than 20% of the cells have complete or partial inhibition of PTP1B and of PTPN2. Preferably, the composition includes greater than 30%, 40%, 50%, 60%, 70%, 80%, 85%, 90%, 91%, 92%, 93%, 94%, 95%, 96%, 97%, 98 or 99% of cells that have complete or partial inhibition of PTP1B. Preferably, the composition includes greater than 30%, 40%, 50%, 60%, 70%, 80%, 85%, 90%, 91%, 92%, 93%, 94%, 95%, 96%, 97%, 98 or 99% of cells that have complete or partial inhibition of PTPN2. In one embodiment, all cells have complete or partial inhibition of PTP1B. In one embodiment, all cells have complete or partial inhibition of PTPN2. In certain embodiments, both PTP1B and PTPN2 are partially inhibited or both are completely inhibited. In further embodiments, PTP1B is partially inhibited and PTPN2 is completely inhibited. In further embodiments, PTP1B is completely inhibited and PTPN2 is partially inhibited.

The present invention also relates to a composition comprising a leukocyte, a PTP1B inhibitor and a PTPN2 inhibitor as described herein. Preferably, the PTP1B inhibitor is an interfering RNA as described herein or a small molecule inhibitor. Preferably the PTPN2 inhibitor is an interfering RNA as described herein, or a small molecule inhibitor. Preferably, the small molecule inhibitor of PTP1B is selected from the group consisting of: claramine, trodusquemine, derivatives thereof (including DPM-1001) or any other small molecule inhibitor described herein. Preferably, the PTPN2 inhibitor is selected from the group consisting of: ethyl-3,4-dephospatin or compound 8 or any other small molecule inhibitor described herein.

The composition may further include a cytokine for enhancing cell killing, such as IL-2 or IFNγ.

In a preferred embodiment, the leukocyte is a CAR T cell, more preferably the CAR-T cell is specific for a cell surface tumour antigen. In one example, the CAR-T cell is specific for HER-2, however it will be appreciated that the method is not limited to the type of tumour antigen expressed by the cancer. In other examples, the CAR-T cell is specific for one or more tumour antigens including but not limited to CD171, EGFR, MSLN, CD19, CD123, Lewis Y, FAP or CD131 or any other tumour antigen.

The T cells may be selected from the group consisting of tumour infiltrating lymphocytes, peripheral blood lymphocyte, genetically engineered to express anti-tumour T cell receptors or chimeric antigen receptors (CARs), γδ T cells, enriched with mixed lymphocyte tumour cell cultures (MLTCs) or cloned using autologous antigen presenting cells and tumour derived peptides. The cells may be isolated from a histocompatible donor, or from the cancer-bearing subject.

In any method of the invention, the leukocytes or T cells are purified or substantially purified prior to culture in the presence of a PTP1B inhibitor and/or PTPN2 inhibitor. This step enriches the leukocytes or T cells by removing other cell types from the biological sample.

In one embodiment, the CAR-T cells are HER-2 specific CAR CD8+ T cells. In alternative embodiments the CAR-T cells are CD19-specific CAR CD8+ T cells, or are CD171-specific CAR CD8+ T cells, or EGFR-specific CAR CD8+ T cells, or CD22-specific CAR CD8+ T cells, or CD123-specific CAR CD8+ T cells, or Lewis Y specific CAR CD8+ T cells, or MSLN-specific CAR CD8+ T cells, or FAP-specific CAR CD8+ T cells, or CD131-specific CAR CD8+ T cells etc. The T cells may be a population that includes more than one type of T cells, comprising any one or more types described herein. For example, the population of T cells may include naïve, activated and/or memory T cells.

In further embodiments, the leukocyte is a NK cell, preferably a CAR NK cell. The CAR may be specific for any cancer antigen, including but not limited to HER-2, CD19, CD171, CD22, CD123, Lewis Y, EGFR, MSLN, FAP and CD131.

The present invention also relates to tumour antigen-specific cytotoxic leukocytes for use in adoptive immunotherapy, the cells comprising a) an exogenous nucleic acid coding an interfering RNA, for example a microRNA, shRNA, siRNA, or gRNA molecule that can reduce the level of PTP1B in a cell and/or b) an exogenous nucleic acid coding an interfering RNA, for example a microRNA, shRNA, siRNA, or gRNA molecule that can reduce the level of PTPN2 in a cell.

The present invention relates to an isolated, purified or recombinant cell comprising an antigen-specific T cell receptor and an exogenous nucleic acid encoding an interfering RNA, for example a microRNA, shRNA, siRNA or gRNA molecule that can reduce the level of PTP1B in a cell, and an exogenous nucleic acid coding an interfering RNA, for example a microRNA, shRNA, siRNA, or gRNA molecule that can reduce the level of PTPN2 in a cell. Preferably, the TCR is specific for a cancer antigen and the cell is a CD8+ T cell. The CD8+ T cell may be a tumour infiltrating lymphocyte or a peripheral blood lymphocyte isolated from a host afflicted with cancer.

In any aspect of the invention, the leukocyte (for example the T cell or NK cell) may be derived from a stem cell, preferably wherein the stem cell is an embryonic stem cell (ESC), embryonic-like stem cell or induced pluripotent stem cell (iPSC). The ESC or iPSC may be differentiated to a leukocyte using any standard technique.

In further embodiments, the leukocyte is obtained by differentiating an ESC or iPSC in vitro to obtain a leukocyte, preferably a T cell or NK cell, that is subsequently subjected to genetic modification to partially, substantially or completely inhibit the activity or level of PTP1B and/or PTPN2 in the leukocyte, preferably a T cell or NK cell.

In still further embodiments, the leukocyte obtained from an ESC or iPSC is subjected to genetic modification to introduce expression of a chimeric antigen receptor (CAR). The genetic modification to introduce expression of a CAR may be before or after the genetic modification to modify expression of the PTPN1 and/or PTPN2 genes. Preferably the CAR is specific for a tumour antigen. The genetic modification to partially, substantially or completely inhibit expression of the PTPN1 and/or PTPN2 genes may be by using a CRISPR-Cas9 RNP system, or any other method for modification of gene expression.

In further aspects, the present invention provides methods increasing the level of T cells in a subject exhibiting an effector memory phenotype, increasing CD8+ T cell mediated immunity for treating a disease in a subject, for forming an immune response in a subject suitable for the treatment of cancer, prolonging the survival of a subject having cancer, or for promoting regression of a cancer in a subject, by administering a cell or composition as described herein.

The present invention therefore also relates to a method for increasing the level of T cells in a subject exhibiting an effector memory phenotype comprising the steps of:
administering a PTP1B inhibitor and a PTPN2 inhibitor to the subject;
thereby increasing the level of T cells in a subject exhibiting an effector memory phenotype.

The present invention relates to a method for increasing the level of T cells in a subject exhibiting an effector memory phenotype comprising the steps of:
culturing a T cell population from a biological sample ex vivo in the presence of a PTP1B inhibitor and a PTPN2 inhibitor;
expanding the cells in culture;
administering the cultured cells to the subject;
thereby increasing the level of T cells in a subject exhibiting an effector memory phenotype.

The present invention also relates to a method of increasing CD8+ T cell mediated immunity in a subject having a disease state comprising:
contacting CD8+ T cells with a PTP1B inhibitor ex vivo for a sufficient time and under conditions to generate a population of CD8+ T cells exhibiting at least one property of a cytotoxic T cell;
administering the population of CD8+ T cells to the subject,
administering a PTPN2 inhibitor to the subject;
thereby increasing CD8+ T cell mediated immunity in a subject.

The present invention also relates to a method of increasing CD8+ T cell mediated immunity in a subject having a disease state comprising:
contacting CD8+ T cells with a PTPN2 inhibitor ex vivo for a sufficient time and under conditions to generate a population of CD8+ T cells exhibiting at least one property of a cytotoxic T cell;
administering the population of CD8+ T cells to the subject,
administering a PTP1B inhibitor to the subject;
thereby increasing CD8+ T cell mediated immunity in a subject.

The present invention also relates to a method of increasing CD8+ T cell mediated immunity in a subject having a disease state comprising:
contacting CD8+ T cells with a PTP1B inhibitor and a PTPN2 inhibitor ex vivo for a sufficient time and under conditions to generate a population of CD8+ T cells exhibiting at least one property of a cytotoxic T cell;
administering the population of CD8+ T cells to the subject,
thereby increasing CD8+ T cell mediated immunity in a subject.

The present invention also relates to a method of increasing CD8+ T cell mediated immunity in a subject having a disease state comprising:
isolating a population of the subject's CD8+ T cells;
introducing a nucleic acid molecule encoding an siRNA, shRNA or gRNA directed to PTP1B into the isolated CD8+ T cells, thereby reducing the level of PTP1B in a CD8+ T cell; and
reintroducing the CD8+ T cells into said subject,
administering a PTPN2 inhibitor to the subject;
thereby increasing the CD8+ T cell mediated immunity in a subject.

The present invention also relates to a method of increasing CD8+ T cell mediated immunity in a subject having a disease state comprising:
isolating a population of the subject's CD8+ T cells;
introducing a nucleic acid molecule encoding an siRNA, shRNA or gRNA directed to PTPN2 into the isolated CD8+ T cells, thereby reducing the level of PTPN2 in a CD8+ T cell; and
reintroducing the CD8+ T cells into said subject,
administering a PTP1B inhibitor to the subject;
thereby increasing the CD8+ T cell mediated immunity in a subject.

The present invention also relates to a method of increasing CD8+ T cell mediated immunity in a subject having a disease state comprising:
isolating a population of the subject's CD8+ T cells;
introducing a nucleic acid molecule encoding an siRNA, shRNA or gRNA directed to PTP1B and an siRNA, shRNA or gRNA directed PTPN2 into the isolated CD8+ T cells, thereby reducing the level of PTP1B and PTPN2 in a CD8+ T cell; and
reintroducing the CD8+ T cells into said subject,
thereby increasing the CD8+ T cell mediated immunity in a subject.

The present invention also relates to a method of increasing CD8+ T cell mediated immunity in a subject having a disease state comprising:
administering a PTP1B inhibitor and a PTPN2 inhibitor to the subject;
thereby increasing CD8+ T cell mediated immunity in a subject.

Preferably the disease state is cancer, more preferably a cancer characterised by the presence of a solid tumour.

The present invention also relates to a method of treating cancer in a subject comprising:
administering a PTP1B inhibitor and a PTPN2 inhibitor to the subject;
thereby treating cancer in the subject.

The present invention relates to a method of prolonging survival of a subject having cancer comprising the steps of:
administering a PTP1B inhibitor and a PTPN2 inhibitor to the subject;
whereupon survival of the subject is prolonged.

The present invention also relates to a method of activating or increasing the number of tumour infiltrating lymphocytes in a subject suffering from cancer, comprising:
administering a PTP1B inhibitor and a PTPN2 inhibitor to the subject;
thereby activating or increasing the number of tumour infiltrating lymphocytes in the subject.

In any embodiment, the tumour infiltrating lymphocytes may be classified as anergic or exhausted lymphocytes.

The present invention also provides a method for forming an immune response in a subject suitable for the treatment of cancer comprising the steps of
administering a PTP1B inhibitor and a PTPN2 inhibitor to the subject;
thereby producing an immune response in a subject suitable for the treatment of cancer.

In one embodiment, the invention provides a method for producing an immune response in a subject suitable for the treatment of cancer, the method comprising the steps of
obtaining T cells from the subject or a histocompatible donor subject;
culturing the T cells in the presence of a PTP1B inhibitor ex vivo for a sufficient time and under conditions for to generate a population of T cells exhibiting at least one cytotoxic T cell property, thereby forming a population of cytotoxic T cells,
administering the population of cytotoxic T cells to the subject,
administering a PTPN2 inhibitor to the subject;
thereby producing an immune response in a subject suitable for the treatment of cancer.

The present invention also provides a method for forming an immune response in a subject suitable for the treatment of cancer comprising the steps of
obtaining T cells from the subject or a histocompatible donor subject;
culturing the T cells in the presence of a PTPN2 inhibitor ex vivo for a sufficient time and under conditions for to generate a population of T cells exhibiting at least one cytotoxic T cell property, thereby forming a population of cytotoxic T cells,
administering the population of cytotoxic T cells to the subject,
administering a PTP1B inhibitor to the subject;
thereby producing an immune response in a subject suitable for the treatment of cancer.

The present invention also provides a method for forming an immune response in a subject suitable for the treatment of cancer comprising the steps of
obtaining T cells from the subject or a histocompatible donor subject;
culturing the T cells in the presence of a PTP1B inhibitor and a PTPN2 inhibitor ex vivo for a sufficient time and under conditions for to generate a population of T cells exhibiting at least one cytotoxic T cell property, thereby forming a population of cytotoxic T cells,
administering the population of cytotoxic T cells to the subject,
thereby producing an immune response in a subject suitable for the treatment of cancer.

The present invention relates to a method of promoting regression of a cancer in a subject comprising the steps of:
culturing T cells obtained from a subject in the presence of a PTPN2 inhibitor;
administering the cultured T cells to the subject;
administering a PTP1B inhibitor to the subject;
whereupon regression of the cancer is promoted.

The present invention relates to a method of promoting regression of a cancer in a subject comprising the steps of:
culturing T cells obtained from a subject in the presence of a PTP1B inhibitor,
administering the cultured T cells to the subject;
administering a PTPN2 inhibitor to the subject;
whereupon regression of the cancer is promoted.

The present invention relates to a method of promoting regression of a cancer in a subject comprising the steps of:
culturing T cells obtained from a subject in the presence of a PTP1B inhibitor and a PTPN2 inhibitor;
administering the cultured T cells to the subject;
whereupon regression of the cancer is promoted.

The present invention relates to a method of promoting regression of a cancer in a subject having cancer comprising the steps of:
culturing CAR-T cells specific for a tumour antigen expressed by the cancer in the presence of a PTP1B inhibitor;
administering the cultured CAR-T cells to the subject,
administering a PTPN2 inhibitor to the subject;
whereupon regression of the cancer is promoted.

The present invention relates to a method of promoting regression of a cancer in a subject having cancer comprising the steps of:
culturing CAR-T cells specific for a tumour antigen expressed by the cancer in the presence of a PTPN2 inhibitor,
administering the cultured CAR-T cells to the subject;
administering a PTP1B inhibitor to the subject;
whereupon regression of the cancer is promoted.

The present invention relates to a method of promoting regression of a cancer in a subject having cancer comprising the steps of:
culturing CAR-T cells specific for a tumour antigen expressed by the cancer in the presence of a PTP1B inhibitor and a PTPN2 inhibitor,
administering the cultured CAR-T cells to the subject,
whereupon regression of the cancer is promoted.

The present invention relates to a method of prolonging survival of a subject having cancer comprising the steps of:
culturing CAR-T cells specific for a tumour antigen expressed by the cancer in the presence of a PTP1B inhibitor;
administering the cultured CAR-T cells to the subject,
administering a PTPN2 inhibitor to the subject;
whereupon survival of the subject is prolonged.

The present invention relates to a method of prolonging survival of a subject having cancer comprising the steps of:
culturing CAR-T cells specific for a tumour antigen expressed by the cancer in the presence of a PTPN2 inhibitor;
administering the cultured CAR-T cells to the subject;
administering a PTP1B inhibitor to the subject;
whereupon survival of the subject is prolonged.

The present invention relates to a method of prolonging survival of a subject having cancer comprising the steps of:
culturing CAR-T cells specific for a tumour antigen expressed by the cancer in the presence of a PTP1B inhibitor and a PTPN2 inhibitor,
administering the cultured CAR-T cells to the subject,
whereupon survival of the subject is prolonged.

In some examples of the above embodiments, the cancer is a HER-2 positive cancer and the CAR-T cell is specific for Her-2, however it will be appreciated that the method is not limited to the type of tumour antigen expressed by the cancer. In other examples, the cancer is positive for the tumour antigens CD171, EGFR, MSLN, CD19, CD123, Lewis Y, FAP, CD22, GD2, or CD131 and the CAR-T cell is specific for any one or more of those antigens.

In any method of the invention, the T cells may not require exposure to a cytokine (such as IL-2, IL-15 or IL-17) prior to being administered to a subject. Alternatively, the individual to whom the T cells are being administered, may not require concomitant administration of a cytokine for enhancing proliferation of the T cells (such as IL-2, IL-15 or IL-17).

In any embodiment of any of the above methods, the PTP1B and/or PTPN2 inhibitor for inclusion or contacting the cells in culture may be the same or a different class of inhibitor. For example, the inhibitors used may both be small molecules, may both be antibodies (e.g., intrabodies), may both be peptides, or peptidomimetics, may both be proteolysis targeting chimeras (PROTACs), may both be TALENs, may both be zinc-finger nucleases, may both be inhibitory or interfering RNAs, such as antisense RNAs, siRNAs, microRNAs, shRNAs, or gRNAs for use in CRISPR-based or other genome editing system. Alternatively, one of the inhibitors (e.g., the PTP1B inhibitor) may be in one class (e.g., a small molecule) and the other inhibitor (e.g., the PTPN2 inhibitor) may be any other class of inhibitor (such as peptide, or peptidomimetic, a PROTAC, an antibody, preferably intrabody, a TALEN, a zinc finger nuclease, an inhibitory or interfering RNA, such as antisense RNA, siRNA, microRNA, shRNA, or gRNA for use in CRISPR-based or other genome editing system). Still further, one of the inhibitors (e.g., the PTPN2 inhibitor) may be in one class (e.g. a small molecule) and the other inhibitor (e.g., the PTP1B inhibitor) may be any other class of inhibitor (such as peptide, or peptidomimetic, a PROTAC, an antibody, preferably intrabody, a TALEN, a zinc finger nuclease, an inhibitory or interfering RNA, such as antisense RNA, siRNA, microRNA, shRNA, or gRNA for use in CRISPR-based or other genome editing system). It will be appreciated that any combination of classes of inhibitor may be used in the methods of the present invention, provided that both PTP1B and PTPN2 are inhibited.

It will also be understood that the contacting of the cell with the PTP1B and PTPN2 inhibitor does not need to be at the same time. For example, in certain embodiments, the cell is contacted with a first PTP1B inhibitor (e.g., an interfering RNA or gRNA for use in a CRISPR-Cas9 genetic modification system) and the contacting with the second PTPN2 inhibitor (e.g., small molecule) is at a later time. Conversely, in other embodiments, the cell is contacted with a first PTPN2 inhibitor (e.g., genome editing or use of an interfering RNA or gRNA for use in a CRISPR-Cas9 genetic modification system) and the contacting with the second PTP1B inhibitor (e.g., small molecule) is at a later time.

Thus it will be appreciated that the inhibition of PTP1B and of PTPN2 does not need to be with the same form of inhibitor, at the same time, or administered via the same route. For example, the inhibition of PTP1B may be a pharmacological inhibition and the inhibition of PTPN2 may be via genome editing (and vice versa).

The present invention relates to a method of treating cancer in a subject comprising administering a population of isolated or purified CD8+ T cells effective to treat the cancer, the CD8+ T cell comprising an antigen-specific T cell receptor, an exogenous nucleic acid encoding an interfering RNA, for example a microRNA, shRNA, siRNA or gRNA molecule, directed to PTP1B and an exogenous nucleic acid encoding an interfering RNA, for example a microRNA, shRNA, siRNA or gRNA molecule, directed to PTPN2.

The present invention relates to a method of promoting regression of a cancer in a subject having cancer comprising the steps of:
administering a PTP1B inhibitor and a PTPN2 inhibitor to the subject;
whereupon regression of the cancer is promoted.

In any embodiment of the invention, the cancer is a HER-2 positive cancer. Alternatively, the cancer may be a CD19 positive cancer, a CD171 positive cancer, an EGFR-positive cancer, a CD22-positive cancer, a CD123-positive cancer, a Lewis Y positive cancer cells, or an MSLN-positive cancer, an FAP-positive cancer, or CD131-positive cancer. It will be appreciated however that the present invention is not limited by the type of cancer requiring treatment.

Further still, in certain embodiments of the invention, the PTP1B and PTPN2 inhibitor is the same molecule. For example, celastrol is a small molecule that inhibits both PTP1B and PTPN2. In certain embodiments, cytotoxic leukocytes are cultured in the presence of an inhibitor which inhibits both PTP1B and PTPN2 prior to administering the cells to a subject in needs thereof. In alternative embodiments, an inhibitor which inhibits both PTP1B and PTPN2 is administered to a subject in need thereof, including after the subject has received a treatment with cytotoxic leukocytes, for example CAR T or CAR NK cells.

In further embodiments, the PTP1B and PTPN2 inhibitor may be the same class/type of molecule. For example, the inhibitors may both be interfering RNAs or gRNAs (or other nuclease-based system) for use in genome editing. Alternatively, the inhibitors may both be small molecules etc.

In any of the above methods, the methods may further include administration of a CAR T cell to the individual. The CAR T cell may be a HER-2 specific CAR CD8+ T cell. In other examples, the CAR T cell is specific for one or more tumour antigens including but not limited to CD171, EGFR, MSLN, CD19, CD123, Lewis Y, FAP or CD131 or any other tumour antigen.

Accordingly, the present invention also relates to a method of treating cancer in a subject comprising:
providing a subject who has received a CAR T cell for the treatment of cancer,
administering a PTP1B inhibitor and a PTPN2 inhibitor to the subject;
thereby treating cancer in the subject.

Further, the present invention relates to a method of enhancing a CAR T therapy for cancer in a subject, the method comprising:
providing a subject who has received a CAR T cell for the treatment of cancer,
administering a PTP1B inhibitor and a PTPN2 inhibitor to the subject,
thereby enhancing the CAR T therapy for cancer in the subject.

The present invention also provides use of a PTP1B inhibitor and a PTPN2 inhibitor in the manufacture of a medicament for:
increasing the level of T cells in a subject exhibiting an effector memory phenotype;
forming an immune response in a subject suitable for the treatment of cancer;
increasing CD8+ T cell mediated immunity in a subject having a disease state;
treating cancer in a subject;
promoting regression of a cancer in a subject having cancer; or
prolonging survival of a subject having cancer.

The medicament may further include CAR T cells. Preferably the CAR T cells are HER-2 specific CAR CD8+ T cells. In other examples, the CAR T cell is specific for one or more tumour antigens including but not limited to CD171, EGFR, MSLN, CD19, CD123, Lewis Y, FAP or CD131 or any other tumour antigen.

The present invention also provides a PTP1B inhibitor and a PTPN2 inhibitor or pharmaceutical composition comprising a PTP1B inhibitor and a PTPN2 inhibitor for use in:
increasing the level of T cells in a subject exhibiting an effector memory phenotype;
forming an immune response in a subject suitable for the treatment of cancer;
increasing CD8+ T cell mediated immunity in a subject having a disease state;
treating cancer in a subject;
promoting regression of a cancer in a subject having cancer; or
prolonging survival of a subject having cancer.

The above use may be in combination with the administration of CAR T cells to an individual requiring treatment. The CAR T cells may be, but are not limited to HER-2 specific CAR CD8+ T cells.

In any aspect of the present invention, the PTP1B inhibitor and/or PTPN2 inhibitor may be administered directly to an individual. The route of administration may be systemic or any route as described herein that allows the inhibitors to enter the circulation. The PTP1B and PTPN2 inhibitors may be administered via the same route or via different routes. Further, the PTP1B and PTPN2 inhibitors may be administered to the subject simultaneously (i.e., in a single dosage form or in two different dosage forms administered at the same time), sequentially in the same intervention, or at separate times whereby the timing of administration of each inhibitor is at least an hour apart, at least several hours apart, at least a day apart, at least several days apart or at least a week or more apart. It will be appreciated that administration of a PTP1B inhibitor or PTPN2 directly to an individual can be used to activate otherwise exhausted tumour infiltrating lymphocytes.

As used herein, a PTP1B inhibitor may be any molecule that inhibits the phosphatase activity of PTP1B. The inhibitor may be a direct inhibitor of the phosphatase active site, may act allosterically to inhibit phosphatase activity, inhibit interaction of PTP1B with its substrate, or may reduce the level of PTP1B by reducing the transcriptional activity of the Ptpn1 gene, or reducing the amount of Ptpn1 mRNA or protein present in the cell.

The PTP1B inhibitor may specifically bind to and directly inhibit PTP1B such that the off-target effects of the PTP1B inhibitor are minimal. Preferably, PTP1B inhibitor inhibits or reduces activity or expression of another target by no more than about 5%, no more than about 10%, no more than about 15%, or no more than about 20%. Preferably, the PTP1B inhibitor inhibits or reduces the activity of PTP1B by at least 50%, at least 60%, at least 70%, at least 80%, at least 90% or more. In certain embodiments, the inhibitor completely inhibits or prevents activity of PTP1B.

Typically, the PTP1B inhibitor is a small molecule, for example claramine, trodusquemine (or the derivative DPM-1001) or any other small molecule inhibitor as described herein, or a peptide, or a peptidomimetic. The PTP1B inhibitor may be an inhibitory antibody, preferably an intrabody for inhibiting PTP1B. The inhibitor may be a PROTAC. The inhibitor may be a TALEN or zinc finger nuclease for use in genome editing for editing part or all of the gene encoding PTP1B. The inhibitor may also be an inhibitory or interfering RNA, such as antisense RNA, siRNA, microRNA, shRNA, or gRNA for use in CRISPR-based or other genome editing system to partially or completed reduce Ptpn1 gene expression.

As used herein, a PTPN2 inhibitor may be any molecule that inhibits the phosphatase activity of PTPN2. The inhibitor may be a direct inhibitor of the phosphatase active site, may act allosterically to inhibit phosphatase activity, inhibit interaction of PTPN2 with its substrate, or may reduce the level of PTPN2 by reducing the transcriptional activity of the Ptpn2 gene, or reducing the amount of Ptpn2 mRNA or protein present in the cell.

The PTPN2 inhibitor may specifically bind to and directly inhibit PTPN2 such that the off-target effects of the PTPN2 inhibitor are minimal. Preferably, PTPN2 inhibitor inhibits or reduces activity or expression of another target by no more than about 5%, no more than about 10%, no more than about 15%, or no more than about 20%. Preferably, the PTPN2 inhibitor inhibits or reduces the activity of PTPN2 by at least 50%, at least 60%, at least 70%, at least 80%, at least 90% or more. In certain embodiments, the inhibitor completely inhibits or prevents activity of PTPN2.

Typically, the PTPN2 inhibitor is a small molecule, for example ethyl-3,4-dephospatin or compound 8 or any other small molecule inhibitor as described herein, or a peptide, or a peptidomimetic. The PTPN2 inhibitor may be an inhibitory antibody, preferably an intrabody for inhibiting PTPN2. The inhibitor may be a PROTAC. The inhibitor may be a TALEN or zinc finger nuclease for use in genome editing. The inhibitor may also be an inhibitory or interfering RNA, such as antisense RNA, siRNA, microRNA, shRNA, or gRNA for use in CRISPR-based or other genome editing system to partially or completed reduce PTPN2 gene expression.

In any aspect of the invention, the only inhibition is of PTP1B and of PTPN2. In other words, no other gene or gene product other than the gene or gene products of Ptp1b and the gene or gene products of Ptpn2 are inhibited. For example, the only genome editing occurs to the Ptp1b and/or Ptpn2 gene or genome editing platform is designed only to target the Ptp1 b and/or Ptpn2 genes. In other words, where the genome editing platform is a TALEN, ZFN or CRISPR/Cas9, or the like, the TALEN, ZFN or gRNA are designed or intended for targeting the Ptp1 b and/or Ptpn2 genes only.

Alternatively, only interfering RNAs that are intended for specifically targeting Ptp1 b and/or Ptpn2 gene expression or which predominantly target Ptpp1b and/or Ptpn2 gene expression are used such that the interfering RNA does not specifically target the expression of any other gene. Further still, and in the alternative, the small molecule inhibitor(s) used, preferably specifically bind(s) to and directly inhibits PTP1B and/or PTPN2 such that the off-target effects are minimal.

In preferred embodiments, the off-target effects (if any) of the inhibitors used is less than 30%, less than 20%, less than 10%, or less than 5% inhibition of a target that is not PTP1B or PTPN2.

In any aspect of the invention, the only phosphatases inhibited are PTP1B and PTPN2.

In any aspect of the invention, both PTP1B and PTPN2 are directly inhibited such that the inhibitors used are for specifically inhibiting PTP1B and/or for specifically inhibiting PTPN2.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. PTP1B-deficiency enhances adoptive CD8⁺ T cell-mediated anti-tumour immunity and prolongs survival. A-E) AT-3-OVA mammary tumour cells (5×10⁵) were injected into the fourth inguinal mammary fat pads of female Ly5.1⁺ mice. Seven days after tumour injection purified 2×10⁶ naïve CD8⁺CD44^{lo}CD62L^{hi} lymph node T cells from Ly5.2⁺;OT-1; Ptp1b^{fl/fl} versus Ly5.2⁺;OT-1;Lck-Cre;Ptp1b^{fl/fl} mice (or vehicle control without T cells) were adoptively transferred into Ly5.1 mice bearing established (40-50 mm²) AT3-OVA mammary tumours and B) tumour growth and C) survival was monitored. D) On day 10 (d10) post adoptive transfer the number of tumour-infiltrating Ly5.2⁺OT-1⁺CD8⁺ T cells were determined by flow cytometry. E) Tumour-infiltrating T cells from (D) were stimulated with PMA/lonomycin in the presence of Golgi Stop/Plug and stained intracellularly for IFN-g and TNF. Intracellular Granzyme B (GrzB) was detected in unstimulated CD8⁺ T cell tumour infiltrates. IFN-g, TNF and GrzB are markers of CD8+ T cell cytotoxicity. Representative results (means±SEM) from at least two independent experiments are shown. In (D, E) significance determined using 2-tailed Mann-Whitney U Test. Significance for tumour sizes in (B) was determined using 2-way ANOVA Test and for survival in (C) using Log-rank (Mantel-Cox) test; ***p<0.001, ****p<0.0001.

FIG. 2. PTP1B-deficiency enhances the tumour-specific activity of HER-2 CAR T cells in vitro. A) CAR T cells were co-cultured with HER-2 expressing 24JK target cells versus HER-2 negative 24JK cells 4 hours prior to analysis and CD25, PD-1 and Lag-3 MFIs on CD8⁺ CAR-T cells were determined by flow cytometry. B) CAR T cells were co-cultured with HER-2 expressing 24JK target cells versus HER-2 negative 24JK at different ratios 4 hours prior to analysis and intracellular IFN-g in CD8⁺ CAR-T cells was determined by flow cytometry. C) CAR-T cells were incubated with 5 mM CTV-labelled (CTV^{bright}) 24JK-HER-2 cells and 0.5 mM CTV-labelled (CTV^{dim}) 24JK sarcoma cells. Antigen-specific target cell lysis (24JK-HER-2 versus 24JK response) was assessed by monitoring for the depletion of CTV^{bright} 24JK-HER-2 cells by flow cytometry. Representative results (means±SEM) from at least two independent experiments are shown. In (A) significance was determined using 2-tailed Mann-Whitney U Test, in (B) was determined using 2-way ANOVA Test; *p<0.05, ***p<0.001, ****p<0.0001.

FIG. 3. Generation of CAR T cells deficient in PTP1B and PTPN2 with CRIPSR/Cas9 RNP genome editing. Flow cytometry of wild-type (C57BL/6), PTP1B-deficient, PTPN2-deficient or PTP1B and PTPN2-deficient CAR T cells stained for CD8 and intracellular PTPN2.

FIG. 4. Combined deletion of PTP1B & PTPN2 ‘supercharges’ CAR T cells by enhancing HER2 CAR T cell activation in vitro. CRISPR-RNP gene editing was used to delete PTPN2 in either control (Ptp1b^{+/+}) or PTP1B-null (Ptp1b^{-/-}) HER-2-specific murine CAR T cells to generate CAR T cells deficient in both PTP1B and PTPN2. The resulting HER2 CAR T cells were then incubated with 24JK-HER-2 versus 24JK sarcoma cells and stained for CD8, intracellular IFNg and TNF. The proportion of CD8⁺IFNg⁺ CAR T cells and CD8⁺TNF⁺ CAR T Cells was determined by flow cytometry. Representative results (means±SEM) from at least two independent experiments are shown. Significance was determined using 2-way ANOVA Test; ****p<0.0001.

FIG. 5. Combined deletion of PTP1B & PTPN2 ‘supercharges’ CAR T cells by enhancing cytotoxicity in vitro. CRISPR-RNP gene editing was used to delete PTPN2 in either control (Ptp1b^{+/+}) or PTP1B-null (Ptp1b^{-/-}) HER-2-specific murine CAR T cells to generate CAR T cells deficient in both PTP1B and PTPN2. The resulting CAR T cells were incubated with 5 mM CTV-labelled (CTV^{bright}) 24JK-HER-2 cells and 0.5 mM CTV-labelled (CTV^{dim}) 24JK sarcoma cells. Antigen-specific target cell lysis (24JK-HER-2 versus 24JK response) was assessed by monitoring for the depletion of CTV^{bright} 24JK-HER-2 cells by flow cytometry.

FIG. 6. Combined deletion of PTP1B & PTPN2 ‘supercharges’ CAR T cells in vivo. A-B) HER-2-E0771 mammary tumour cells (2×10⁵) were injected into the fourth inguinal mammary fat pads of female HER-2 TG mice. Seven days after tumour injection HER-2 TG mice received total body irradiation (4 Gy) followed by the adoptive transfer of total 5×10⁶ HER-2 CAR T cells (=0.8×10⁶ viable mCherry⁺HER2 CAR⁺CD8⁺CD44^{hi}CD62L^{hi} T cells) control (Ptp1b+/+) or PTP1B-null (Ptp1b^{-/-}) HER-2 CAR T cells transfected with control (Ctrl) or Ptpn2-specific sgRNAS to delete PTPN2 by CRISPR-RNP; mice were not administered IL-2. A) HER-2 mice were monitored for tumour growth and B) tumour weights and CD45⁺CD8⁺mCherry⁺ CAR T cell infiltrates in tumours and spleen determined by flow cytometry. Significance in (A) was determined using 2-way ANOVA Test; ****p<0.0001.

FIG. 7. CRISPR-Cas9/RNP-Mediated PTP1B deletion in human T cells enhances TCR-mediated activation and proliferation. A-D) CRISPR RNP was used to delete PTP1B in human PBMC-derived T cells from four individual donors [PBMCs stimulated with a-CD3 (OKT3) and IL-2 for 72 h] and were processed for A) immunoblotting, B) intracellular p-STAT-5, Bcl-xL or Bcl-2 (MFIs) analysis by flow cytometry, or C) re-stimulated with a-CD3 overnight for the analysis of CD69 (MFIs) by flow cytometry. D) Alternatively, CTV-labelled control and PTP1B-deficient PBMC-derived human T cells were stimulated with plate-bound a-CD3 (OKT3) for 5 days and T cell proliferation (CTV dilution) assessed by flow cytometry. Representative results (means±SEM) from at least two independent experiments are shown. In (B-D) significance was determined using 1-way ANOVA Test; *p<0.05, **p<0.01.

FIG. 8: PTP1B-deficiency enhances the tumour-specific activity of HER2 CAR T cells in vivo. A-C) HER-2-E0771 mammary tumour cells (2×10⁵) were injected into the fourth inguinal mammary fat pads of female HER-2 TG mice. Seven days after tumour injection HER-2 TG mice received total body irradiation (4 Gy) followed by the adoptive transfer of total 20×10⁶ HER-2 CAR T cells (=6×10⁶ viable mCherry⁺HER2 CAR⁺CD8⁺CD44^{hi}CD62L^{hi} T cells) generated from Ptp1b^{fl/fl} versus Lck-Cre;Ptp1b^{fl/fl} splenocytes and monitored for B) tumour growth and c) survival. Representative results (means±SEM) from at least two independent experiments are shown. Significance in b) was determined using 2-way ANOVA Test and in C) using Log-rank (Mantel-Cox) test; ****p<0.0001.

FIG. 9: CRISPR-Cas9/RNP-Mediated PTP1B deletion in human Lewis Y (LY) CAR T cells enhances the generation of central memory CAR T cells and promotes CAR T cell activation. A-E) CRISPR RNP was used to delete PTP1B in human PBMC-derived LY CAR T cells from four individual donors (PBMCs stimulated with OKT3 and IL-2 for 72 h and then transduced with a retrovirus encoding a CAR consisting of an extracellular scFv-anti-human LeY domain, a membrane proximal CD8 hinge region and the transmembrane and the cytoplasmic signaling domains of CD28 fused to the cytoplasmic region of CD3z. LY CAR T cells were processed for A) immunoblotting, or B) stained with fluorophore-conjugated antibodies to determine the frequency of CD8⁺LY⁺CD45RO⁺CD62L⁺ central memory CAR T cells. C-E) Alternatively, CD8⁺LY⁺ CAR T cells were incubated with LY-negative MDA-MB-435 cells and LY-expressing OVCAR-3 cells for the analysis of C) CD69 mean fluorescence intensity (MFI), D) Tim-3 MFI or E) intracellular TNF by flow cytometry. Representative results (means±SEM) from at least two independent experiments are shown. In (B) significance was determined using 1-way ANOVA Test, in (c-d) using 2-way ANOVA Test; *p<0.05, **p<0.01.

FIG. 10: PTP1B-inhibition with MSI-1436 enhances the tumour-specific activity of HER2 CAR T cells in vivo. HER-2-E0771 mammary tumour cells (2×10⁵) were injected into the fourth inguinal mammary fat pads of female HER-2 TG mice. Seven days after tumour injection HER-2 TG mice received total body irradiation (4 Gy) followed by the adoptive transfer of total 20×10⁶ HER-2 CAR T cells (=6×10⁶ viable mCherry⁺HER2 CAR⁺CD8⁺CD44^{hi}CD62L^{hi} T cells) generated from Ptp1b^{fl/fl} versus Lck-Cre;Ptp1b^{fl/fl} splenocytes; mice were not administered IL-2. Mice were treated with PTP1B specific allosteric inhibitor MSI-1436 (5 mg/kg intraperitoneally) or saline on days 1, 4, 7, 10, 13, 16 and 19 post adoptive transfer and tumour growth was monitored. Representative results (means±SEM) from at least two independent experiments are shown. Significance was determined using 2-way ANOVA Test; *p<0.05 ****p<0.0001.

FIG. 11: Combined deletion of PTP1B & PTPN2 in human Lewis Y (LY) CAR T cells further enhances the CAR T cell activation. CRISPR RNP was used to delete PTP1B and PTPN2 in human PBMC-derived LY CAR T cells from 3 individual donors (PBMCs stimulated with OKT3 and IL-2 for 72 h and then transduced with a retrovirus encoding a CAR consisting of an extracellular scFv-anti-human LeY domain, a membrane proximal CD8 hinge region and the transmembrane and the cytoplasmic signaling domains of CD28 fused to the cytoplasmic region of CD3z. LY⁺ CAR T cells were incubated withLY-negative MDA-MB-435 cells and LY-expressing OVCAR-3 cells and intracellular TNF in CD8⁺LY⁺ CAR T cells was determined by flow cytometry. Representative results (means±SEM) from at least two independent experiments are shown.

FIG. 12: Deletion of PTPN2 & inhibition of PTP1B ‘supercharges’ CAR T cells in vivo. HER-2-E0771 mammary tumour cells (2×10⁵) were injected into the fourth inguinal mammary fat pads of female HER-2 TG mice. Seven days after tumour injection HER-2 TG mice received total body irradiation (4 Gy) followed by the adoptive transfer of total 5×10⁶ HER-2 CAR T cells (=0.8×10⁶ viable mCherry⁺HER-2 CAR⁺CD8⁺CD44^{hi}CD62L^{hi} T cells) control (ctrl) or Ptpn2^{-/-} HER-2 CAR CD8 T cells (PTPN2 had been deleted by CRISPR-RNP). HER-2 mice were treated with MSI-1436 (5 mg/kg intraperitoneally) or saline on days 1, 4, and 7 post adoptive transfer and tumour growth was monitored. Significance in was determined using 2-way ANOVA Test; ****p<0.0001.

FIG. 13: PTP1B-deficiency enhances NK (natural killer) cell-mediated anti-tumour immunity. AT-3-OVA mammary tumour cells (5×10⁵) were injected into the fourth inguinal mammary fat pads of female Ptpn1^{fl/fl} or NK cell specific PTP1B-deficient Ncr1-Cre;Ptpn1^{fl/fl} mice and tumour growth was monitored. Significance was determined using 2-way ANOVA Test; ****p<0.0001.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The inventors have developed a method for the efficient preparation of cells for use in adoptive cell transfer, particularly for cancer immunotherapy. The inventors have surprisingly found that simultaneously inhibiting the activity of PTP1B and PTPN2 in T cells enhances the activation of such cells and their capacity for killing a target cell. Further, an advantage of the present invention is that T cells which are tolerised but would otherwise be useful in adoptive cell transfer (ADC), for example as they are specific for tumour antigens in the case of tumour infiltrating lymphocytes, can be reinvigorated and tolerance reduced. A further advantage of a method of the present invention is that T cells can be differentiated down the cytotoxic CD8+ T cell lineage ex vivo without the need for the presence of CD4+ T cell help.

Still further, the inventors believe that inhibition of PTP1B and PTPN2 in T cells substantially reduces the need for concomitant stimulation with cytokines (for example, to enhance expansion of the cells intended for ADC). Without wishing to be bound by theory, the inventors believe that cells for ADC which are also treated to inhibit PTP1B and PTPN2 activity are more sensitive to cytokines such as IL-17, IL-15 and IL-2 so that fewer cells can be used for ADC, given the increased responsiveness of T cells to cytokines when PTP1B and PTPN2 are inhibited.

Without being bound by any theory or mode of action, it is believed that inhibition of PTP1B and PTPN2 activity causes alteration in T cell receptor (TCR) signalling thereby reversing or avoiding tolerance and instead promoting differentiation of T cells down the cytotoxic T cell lineage. For example, isolated CD8+ T cells are treated so as to reduce PTP1B and PTPN2 activity lead to any one or more of the following functions: development of cytotoxic activity towards cells that bear an antigen to which an enhanced immune response would be desirable, enhanced sustenance and/or antigen-recall responses to presentation of the antigen, or have functional and/or phenotypic characteristics of effector T cells.

Although cancer immunotherapies of ex vivo cultured CD8+ T cells have been demonstrated to exhibit remarkable efficacy, such therapies are not effective in every patient as it is difficult to obtain an effective number of CD8+ T cells that have the ability to target the tumour cells and kill the tumour cell once recognised. The present invention provides a means for producing cells that have an enhanced capacity to kill a target cell, such as a tumour cell.

The inventors believe that a significant advantage of targeting PTP1B and PTPN2 is that this approach not only drives TCR or CAR signalling, but also IL-2-STAT5 signalling, thereby overcoming tolerisation and increasing antigen-induced cytotoxicity. The inventors believe that deletion of PTPN2 drives STAT5 signalling, CXCR3 expression and homing to CXCL9/10/11 expressing tumours, thereby overcoming a major hurdle for CAR T cell therapy of solid tumours. This, coupled with findings that PTPN2 deletion drives STAT1 signalling to overcome T cell exhaustion in the tumour microenvironment, and that PTP1B deletion enhances T cell survival and markedly expands T cells in vivo, is expected to deliver improved treatment efficacy.

A further advantage identified by the inventors is that inhibition of PTP1B in T cells increases persistence of central memory and effector memory T cells. This means that in addition to providing for an increase in cytotoxic killing in the period immediately after PTP1B inhibition, the methods of the present invention provide for better adaptation and preparation of the immune system to deal with long term or subsequent exposure to a relevant antigen (for example, upon relapse of the relevant disease or condition).

The inventors have shown that inhibition of both PTP1B and PTPN2 in CAR T cells enhances tumour-specific responses as well as dramatically enhancing tumour-specific lysis by the T cells. Thus, by targeting both of these phosphatases in CAR T cells, the inventors have identified an approach that enhances the efficacy of CAR T mediated immunotherapy.

Importantly, the inventors have obtained data indicating that targeting of both PTPN1 and PTPN2 results in a synergistic effect on CAR T cell cytotoxicity as reflected by the markedly increased antigen-induced TNF and IFNγ and killing capacity in vitro. This leads to markedly increased effects in vivo models such that tumours are rapidly cleared.

Cells

The present invention includes various methods for culturing, modifying and administering cytotoxic leukocytes to a subject in need thereof. It will be understood that a cytotoxic leukocyte includes any leukocyte that has cell killing (i.e., cytotoxic) properties. Examples of cytotoxic leukocytes include CD8+ T cells, B cells, Natural Killer (NK) cells or proinflammatory monocytes. Preferably, the leukocytes comprise T cells including CD4+ and CD8+ T cells. The T cells may also include effector and effector memory T cells and/or central memory T cells. The leukocytes (preferably T cells or NK cells) may also be genetically engineered to express anti-tumour T cell receptors or chimeric antigen receptors (CARs), or may be γδ (gamma/delta) T cells. The leukocytes may also comprise tumour infiltrating lymphocytes, peripheral blood lymphocyte, or be enriched with mixed lymphocyte tumour cell cultures (MLTCs) or cloned using autologous antigen presenting cells and tumour derived peptides.

Anatomic sources of leukocytes from a subject include peripheral blood, tumours, malignant effusions, and draining lymph nodes. Lymphocytes used for adoptive transfer can either be derived from the stroma of resected tumours (tumour infiltrating lymphocytes), or from blood and: genetically engineered to express antitumour T cell receptors or chimeric antigen receptors (CARs), enriched with mixed lymphocyte tumour cell cultures (MLTCs) or cloned using autologous antigen presenting cells and tumour derived peptides.

The lymphocytes used for infusion can be isolated from an allogenic donor, preferably HLA matched, or from the cancer-bearing subject. In one embodiment, the T cells can be from a healthy individual. In one embodiment, the leukocytes, preferably T cells, from a subject are not obtained or derived from the bone marrow.

Further still, the leukocytes can be derived from stem cells, including an induced pluripotent stem cell (iPSC), or from fetal stem cells (embryonic stem cells or ESCs). Methods for differentiating iPSCs or ESCs to various cell fates (including T cell or NK cells) are replete within the art and will be known to the skilled person. The iPSC may be derived from the cells of the subject requiring treatment. More specifically, the iPSC may be derived from a somatic cell that has been obtained from the subject requiring treatment, and subjected to reprogramming towards a pluripotent state. Again, methods for reprogramming somatic cells to a pluripotent state (i.e., generation of iPSCs) are replete within the art. Non-limiting examples of suitable methods for reprogramming somatic cells are replete in the art, and are exemplified in WO 2009/101407, WO 2014/200030, WO 2015/056804, WO 2014/200114, WO 2014/065435, WO 2013/176233, WO 2012/060473, WO 2012/036299, WO 2011/158967, WO 2011/055851, WO 2011/037270, WO 2011/090221, the contents of which are hereby incorporated by reference.

Alternatively, the iPSC may be derived from an allogenic donor, preferably HLA matched. Further still the ESCs may be obtained from the donor requiring treatment or from an allogeneic donor.

iPSCs can also be used to generate leukocytes expressing a chimeric antigen receptor (CAR). For example, the leukocyte may be an iPSC-derived CAR-expressing T cell whereby iPSCs are genetically modified to express a CAR prior to differentiation to a T cell, NK cell or the like. Methods for generating such cells are known in the art, for example in Themeli et al., (2013), Nature Biotechnology, 31: 928-933, incorporated herein by reference.

In any method of the invention the leukocytes, preferably T cells or NK cells that have been cultured in the presence of a PTP1B inhibitor and/or PTPN2 inhibitor can be transferred into the same mammal from which cells were obtained. In other words, the cells used in a method of the invention can be an autologous cell, i.e., can be obtained from the mammal in which the medical condition is treated or prevented. Alternatively, the cell can be allogenically transferred into another subject. Preferably, the cell is autologous to the subject in a method of treating or preventing a medical condition in the subject.

One source of T cells or NK cells targeted for cancer immunotherapy may be to use artificial chimeric receptors derived, for example, from the antigen binding domain of a monoclonal antibody. When coupled to appropriate intracellular signaling domains, T cells or NK cells expressing these chimeric antigen receptors (CAR) can kill tumour cell targets. CAR T cells have the advantage of acting in a MHC unrestricted manner, allowing them to target tumour cells in which antigen processing or presentation pathways are disrupted. Moreover, they can be directed to nonpeptide antigens on the cell surface, broadening the range of target structures that can be recognized on malignant cells. Hence, CAR-expressing T cells can complement MHC restricted cytotoxic T cells, and increase the overall effectiveness of this cellular immunotherapy.

When naive CD8+ and CD4+ T cells engage peptide antigen presented by major histocompatibility complex (MHC) molecules, the T cell receptor signal strength determines whether T cells progress past the G1 restriction point and commit to cellular division, produce interleukin-2 (IL-2) and undergo clonal expansion/proliferation and differentiate and acquire various effector functions. TCR signaling is reliant on tyrosine phosphorylation mediated by the Src family protein tyrosine kinases, Lck and Fyn, and the Syk family PTK ZAP-70. Engagement of the TCR allows for Lck to phosphorylate the immunoreceptor tyrosine-based activation motifs of the TCR that result ZAP-70 recruitment and activation and the phosphorylation of adaptor proteins such as LAT. This in turn allows for the nucleation of signaling complexes and the phosphorylation and activation of multiple effector pathways. Upon TCR engagement, the activation and/or functions of Lck are regulated by the localisation of Lck and its substrates, as well as the abundance, activity and segregation of regulatory molecules within the immunological synapse. Such regulatory molecules include protein tyrosine phosphatases (PTPs) that regulate the phosphorylation of the Lck Y505 inhibitory site, as well as the Lck Y394 activating site.

PTP1B and PTP1B Inhibitors

PTP1B (also known as PTPN1, PTP1B, protein tyrosine phosphatase, non-receptor type 1, Tyrosine-protein phosphatase non-receptor type 1 or protein-tyrosine phosphatase 1B, encoded by the Ptpn1 gene) is a ubiquitous phosphatase anchored in the endoplasmic reticulum by its C-terminal end and has its catalytic regions exposed to the cytosol. PTP1B is known to dephosphorylate a wide variety of phosphoproteins, such as receptors for the growth factors insulin and epidermal growth factor (EGF), c-Src and beta-catenin. PTP1B also dephosphorylates Janus-activated protein kinase 9JAK) family members including Tyk-2 and JAK-2. PTP1B is reported to be a major negative regulator of the insulin receptor and also of leptin signalling. The PTPN1 gene, which encodes PTP1B, is located in 20q13, a genomic region that is linked to insulin resistance and diabetes in human populations from different geographical origins. More than 20 single nucleotide polymorphisms (SNPs) that are associated with increased risk of type 2 diabetes have been identified within the Ptpn1 gene. Whole-body deletion of PTP1B in mice results in increased insulin sensitivity and improved glucose tolerance. In addition, PTP1B has been shown to modulate cytokine receptor signalling, including IFN-γ signalling. The role of PTP1B in cancer is unclear, with either increased or reduced expression observed in different cancer types.

In order to determine if the presence of a PTP1B inhibitor has inhibited PTP1B, experiments such as the following could be performed: measure PTP1B activity in PTP1B immunoprecipitates using p-NPP (para-nitrophenylphosphate) and p-tyr-RCML (p-tyr-reduced, carboxyamidomethylated and maleylated lysozyme) as substrates as described previously (Bukczynska P et al. Biochem. J. 2004 Jun. 15; 380(Pt 3):939-49; Tiganis T et al. J. Biol. Chem. 1997 Aug. 22; 272(34):21548-57). Alternatively, analysis of known substrates of PTP1B such as c-Src, insulin receptor, EGF receptor, Tyk-2, JAK-2 and the transcription factor STAT5 for tyrosine-phosphorylation by flow cytometry and immuno-blotting can be performed.

As used herein, a “compound that inhibits PTP1B”, or an “PTP1B inhibitor” or “inhibitor of PTP1B” is any compound that inhibits the activity of PTP1B, for example, completely or partially reduces one or more functions of PTP1B including those as described herein. Inhibition of activity of PTP1B may also include a reduction in the level or amount of PTP1B protein, RNA or DNA in a cell. The compound may be a competitive, non-competitive, orthosteric, allosteric, or partial inhibitor. In a preferred form the compound is a molecule that inhibits the enzyme activity.

A PTP1B inhibitor useful in the present invention is one that completely or partially reduces one or more functions of PTP1B as described herein. Preferably, a PTP1B inhibitor reduces phosphatase activity of PTP1B (such as a small molecule, peptide or peptidomimetic, antibody/intrabody or PROTAC), reduces the transcriptional activity of the Ptp1b gene, or reduces the amount of PTP1B mRNA or protein present in the cell.

As used herein, an intrabody is an antibody that has been designed to be expressed intracellularly and can be directed to a target antigen in various subcellular locations.

In any embodiment of the invention, the inhibition of PTP1B may be inhibition of at least 50%, at least 60%, at least 70%, at least 80%, at least 90% or 100% inhibition.

In further embodiments, the inhibition is of PTP1B only, such that there are minimal-to-no off-target effects resulting in inhibition of other targets. Accordingly, in preferred embodiments, the inhibition of targets other than PTP1B, by the PTP1B inhibitor, is no more than 20%, no more than 10%, no more than 5% inhibition.

As used herein, a PTP1B inhibitor may be any molecule that inhibits the phosphatase activity of PTP1B or reduces the level of PTP1B in a cell. The inhibitor may be a direct inhibitor of the phosphatase active site, may act allosterically to inhibit phosphatase activity, inhibit interaction of PTP1B with its substrate, or may reduce the level of PTP1B by reducing the transcriptional activity of the PTP1B gene, or reducing the amount of PTP1B mRNA or protein present in the cell.

An example of a direct inhibitor of the phosphatase active site, an inhibitor that acts allosterically to inhibit phosphatase activity, or an inhibitor that inhibits interaction of PTP1B with its substrate is a small molecule, for example:
Claramine (Sigma, 1545; also referred to as (3β,6β)-6-[[3-[[4-[(3-Aminopropyl)amino]butyl]amino]propyl]amino]-cholestan-3-ol) and derivatives thereof;
Trodusquemine (MSI-1436, produlestan, Trodulamine, troduscemine, CAS No: 186139-09-3, a naturally-occurring cholestane and non-competitive, allosteric inhibitor of PTP1B, trodusquemine selectively targets and inhibits PTP1B, thereby preventing PTP1B-mediated signalling) and derivatives thereof including DPM-1001 (Krishnan et al 2018, JBC, 293:1517-1525);
3-(3,5-dibromo-4-hydroxy-benzoyl)-2-ethyl-benzofuran-6-sulfonicacid-(4-(thiazol-2-ylsulfamyl)-phenyl)-amide (also referred to as PTP Inhibitor XXII, CAS no: 765317-72-4, Thermofisher Scientific or Calbiochem) and derivatives thereof;
3-Hexadecanoyl-5-hydroxymethyl-tetronic acid calcium salt (RK-682, CAS no: 332131-32-5, Santa Cruz Biotechnology) and derivatives thereof;
2-[(Carboxycarbonyl)amino]-4,5,6,7-tetrahydrothieno[2,3-c]pyridine-3-carboxylic acid hydrochloride (TCS-401, CAS no: 243966-09-8, Santa Cruz Biotechnology) and derivatives thereof;
6-Methyl-2-(oxalylamino)-4,5,6,7-tetrahydrothieno[2,3-c]pyridine-3-carboxylic acid trifluoroacetic acid salt (BML-267, Santa Cruz Biotechnology) and derivatives thereof; or a peptide, or peptidomimetic.

As used herein, reference to PTP1B inhibitor or inhibitor of PTP1B also includes a pharmaceutically acceptable salt, ester, polymorph or prodrug thereof.

The inhibitor may also be an antibody such as a monoclonal antibody, preferably wherein the antibody is an intrabody.

In further embodiments, the inhibitor may be a PROTAC which targets PTP1B for degradation.

A PROTAC is a chimeric construct which is useful for facilitating intracellular degradation of a target protein. To facilitate a protein for degradation by the proteasome (eg. degradation of PTP1B), the PROTAC is comprised of a first moiety that binds to an E3 ubiquitin ligase and a second moiety that binds to PTP1B. These moieties are typically connected with a linker. The PROTAC brings the E3 ubiquitin ligase in proximity with the protein so that it is ubiquitinated and marked for degradation. The moiety of a PROTAC for binding to PTP1B can be any peptide, small molecule or antibody, preferably intrabody, that binds to PTP1B. Methods for generating PROTACs, including small-molecule, peptide-based PROTACs and PROTAC-antibody conjugates are known in the art (see for example, GB 2554071, WO 2018051107, WO 2016146985, WO2017/201449 and Zou et al., (2019), Cell Biochem Funct, 37: 21-30).

An example of an inhibitor that may reduce the amount of PTP1B mRNA or protein present in the cell is an inhibitory or interfering RNA, such as antisense RNA, siRNA, microRNA or shRNA.

An example of an shRNA sequence which may reduce the amount of PTP1B mRNA include:

(SEQ ID NO: 1)
AATTGCACCAGGAAGATAATGACTATATC

Exemplary siRNA sequences include:

Sense:
(SEQ ID NO: 2)
′5-UAGGUACAGAGACGUCAGUdTdT-3′;
Antisense:
(SEQ ID NO: 3)
5′-ACUGACGUCUCUGUACCUAdTdT-3
Sense,
(SEQ ID NO: 4)
5′-UAGGUACAGAGACGUCAGUdTdT-3′;
Antisense,
(SEQ ID NO: 5)
5′-ACUGACGUCUCUGUACCUAdTdT-3′
Sense,
(SEQ ID NO: 6)
5-′AAATCAACGGAAGAAGGGTCT-3′
Sense:
(SEQ ID NO: 7)
5′-NNUGACCAUAGUCGGAUUAAA-3′
Sense:
(SEQ ID NO: 8)
5′-UUGAUGUAGUUUAAUCCGACUAUGG-3′
Anti-sense:
(SEQ ID NO: 9)
5′-CCAUAGUCGGAUUAAACUACAUCAA-3′

The skilled person will also appreciate that it is possible to obtain shRNAs or siRNAs, which can be used to reduce PTP1B mRNA, from a number of commercial sources, including from Dharmacon (Madrid, Spain) and Thermofisher (USA). Commercially available shRNA targeted to ptp1b can be purchased, for example, from Open Biosystems (Dharmacon) under catalog no. RHS3979-9571385.

Preferably, the siRNA, shRNA target is (GenBank NCBI Reference Sequences referred to):
exon 2, preferably starting at position 291 of NM_001278618.1;
exon 3, preferably starting at position 382 of NM_002827.3;
exons 3 and 4, preferably starting at position 466 of NM_001278618.1;
exons 4 and 5, preferably starting at position 557 of NM_002827.3; or
exons 2 and 3, preferably starting at position 360 of NM_002827.3.

Preferably, the shRNA has a sequence of at least 50%, 60%, 70%, 80%, 90%, 91%, 92%, 93%, 94%, 95%, 96%, 97%, 98% or 99% identity to any sequence described herein provided the shRNA still retains the ability to reduce PTP1B levels in a cell.

Other sequences include:

TRCN0000350332, with a target sequence of TTTGACCATAGTCGGATTAAA (SEQ ID NO: 87) beginning at position 299 of PTPN1 sequence from NM_002827.4 and a hairpin sequence of:

SEQ ID NO: 10;
5′CCGGTTTGACCATAGTCGGATTAAACTCGAGTTTAATCCGACTATG
GTCAAATTTTTG3′;

TRCN0000010740/TRCN0000350331, with a target sequence of CTGTGATCGAAGGTGCCAAAT (SEQ ID NO: 88) beginning at position 963 of PTPN1 sequence from NM_001278618.1, NM_002827.4 and a hairpin sequence of:

SEQ ID NO: 11;
5′CCGGCTGTGATCGAAGGTGCCAAATCTCGAGATTTGGCACCTTCGA
TCACAGTTTTTG3′;

TRCN0000002778, with a target sequence of CCTAACACATGCGGTCACTTT (SEQ ID NO: 89) beginning at position 410 of PTPN1 sequence from NM_001278618.1, NM_002827.4 and a hairpin sequence of:

SEQ ID NO: 12;
5′CCGGCCTAACACATGCGGTCACTTTCTCGAGAAAGTGACCGCATGT
GTTAGGTTTTTG3′;

TRCN0000002780, with a target sequence of TGCGACAGCTAGAATTGGAAA (SEQ ID NO: 90) beginning at position 609 of PTPN1 sequence from NM_001278618.1, NM_002827.4 and a hairpin sequence of:

SEQ ID NO: 13;
5′CCGGTGCGACAGCTAGAATTGGAAACTCGAGTTTCCAATTCTAGCT
GTCGCATTTTTG3′;

TRCN0000002780/TRCN0000320522, with a target sequence of TGCGACAGCTAGAATTGGAAA (SEQ ID NO: 90) beginning at position 609 of PTPN1 sequence from NM_001278618.1, NM_002827.4 and a hairpin sequence of:

SEQ ID NO: 14;
5′CCGGTGCGACAGCTAGAATTGGAAACTCGAGTTTCCAATTCTAGCT
GTCGCATTTTTG3′;

TRCN0000002779/TRCN0000320521, with a target sequence of GAAGCCCAAAGGAGTTACATT (SEQ ID NO: 91) beginning at position 371 of PTPN1 sequence from NM_001278618.1, NM_002827.4 and a hairpin sequence of:

SEQ ID NO: 15;
5′CCGGGAAGCCCAAAGGAGTTACATTCTCGAGAATGTAACTCCTTTG
GGCTTCTTTTTG3′;

TRCN0000002777/TRCN0000320590, with a target sequence of GCTGCTCTGCTATATGCCTTA (SEQ ID NO: 92) beginning at position 3203 of PTPN1 sequence from NM_001278618.1, NM_002827.4 and a hairpin sequence of:

SEQ ID NO: 16;
5′CCGGGAAGCCCAAAGGAGTTACATTCTCGAGAATGTAACTCCTTTG
GGCTTCTTTTTG3′;

Further, the inhibition of PTP1B may also include genome editing to delete or modify all or part of a sequence encoding PTP1B. The genome editing may be a modification that includes an insertion, deletion, integration of sequence modification/substitution such that the expression of functional PTP1B protein is reduced or ablated. Genome editing techniques are well known in the art and include the use of various nucleases including TALENs, zinc finger nucleases and meganucleases.

The inhibitor may therefore be in the form of a compound/molecule for use in genome editing to remove or modify all or part of a sequence encoding PTP1B. In one example, the genome-editing molecule may be a TALEN, meganuclease or a zinc-finger nuclease which is specifically designed to remove or modify all or part of a sequence encoding PTP1B.

Another exemplary genome editing technique is the CRISPR/Cas9 system (Jinek, M., et al. (2012) Science, 337, 816-821; Cong L., et al. (2013) Science, 339, 819-823; and Qi, L. S., et al. (2013) Cell, 152, 1173-1183), and related technology including CRISPR/Cas12a, CRISPR/Cas13. As such, in accordance with the present invention, the PTP1B inhibitor may include a gRNA (including an sgRNA) for use in CRISPR-related genome editing to inhibit or delete PTP1B activity. More specifically, the present invention contemplates the use of CRISPR-Cas9 to delete Ptpn1 in human CAR T cells. Moreover, use of CRISPR-Cas9 enables the inhibition to be of PTP1B alone (i.e., wherein only PTP1B is inhibited). In certain embodiments, the inhibition of only PTP1B may be complete inhibition (i.e., knock-out) of PTP1B function, or a reduction in PTP1B/Ptpn1 activity/expression (i.e., knock-down or partial knock-out).

The skilled person will be able to purchase or design gRNAs or crRNAs which target a variety of PTP1B sequences. Examples of such gRNA target sequences include:

(SEQ ID NO: 17)
TTCGAGCAGATCGACAAGTC
(SEQ ID NO: 18)
GATGTAGTTTAATCCGACTA
(SEQ ID NO: 19)
GAGCTGGGCGGCCATTTACC
(SEQ ID NO: 20)
TGACGTCTCTGTACCTATTT
(SEQ ID NO: 21)
CAAAAGTGACCGCATGTGTT
(SEQ ID NO: 22)
GTCTTTCAGTTGACCATAGT
(SEQ ID NO: 23)
GGTAAGAATGTAACTCCTTT
(SEQ ID NO: 24)
GGGTAAGAATGTAACTCCTT
(SEQ ID NO: 25)
GATGTAGTTTAATCCGACTA
(SEQ ID NO: 26)
GTGTGGGAGCAGAAAAGCAG
(SEQ ID NO: 27)
GGTGTGGGAGCAGAAAAGCA
(SEQ ID NO: 28)
GAGAAAGGTTCGGTAAGTCT
(SEQ ID NO: 29)
GACCGCATGTGTTAGGCAAA
(SEQ ID NO: 30)
GGCCCTTTGCCTAACACATG
(SEQ ID NO: 31)
GTCTTTCAGTTGACCATAGT
(SEQ ID NO: 32)
GTCACTTTTGGGAGATGGTG
(SEQ ID NO: 33)
GGAAGTCACTGGCTTCATGT
(SEQ ID NO: 34)
GAAGCTTGGCCACTCTACAT
(SEQ ID NO: 35)
GGAAGCTTGGCCACTCTACA
(SEQ ID NO: 36)
GCTATGTGTTGCTGTTGAAC
(SEQ ID NO: 37)
GTGCACTGCAGTGCAGGCAT
(SEQ ID NO: 38)
GGTCACTCAGCCCGGAGCAC
(SEQ ID NO: 39)
GTTGTGGTGCACTGCAGTGC
(SEQ ID NO: 40)
GGCTGAGTGACCCTGACTCT
(SEQ ID NO: 41)
GATTCAGGGACTCCAAAGTC
(SEQ ID NO: 42)
GACTCCAAAGTCAGGCCATG
(SEQ ID NO: 43)
GGCTGATACCTGCCTCTTGC
(SEQ ID NO: 44)
GCTGGTAAGGAGGCCCTCGC
(SEQ ID NO: 45)
GTAGGAGAAGCGCAGCTGGT
(SEQ ID NO: 46)
GAAGGTGCCAAATTCATCAT
(SEQ ID NO: 47)
GAAATGAGGAAGTTTCGGAT
(SEQ ID NO: 48)
GGTGAAGGAAGAGACCCAGG
(SEQ ID NO: 49)
GTTCTTCCCAAATCACCAGT
(SEQ ID NO: 50)
GCTGCTCTTTCAAGGATCAG
(SEQ ID NO: 51)
GGTGGGGGGATATGCTCGGG
(SEQ ID NO: 52)
GGGTCTCTTCCTTCACCCAC
(SEQ ID NO: 53)
GGAGCTTTCCCACGAGGACC
(SEQ ID NO: 54)
GGCTCCAGGATTCGTTTGGG
(SEQ ID NO: 55)
GGATAAAGACTGCCCCATCA
(SEQ ID NO: 56)
GGAAACATACCCTGTAGCAG
(SEQ ID NO: 57)
GTTAGAAGTCGGGTCGTGGG
(SEQ ID NO: 58)
GGAGCCGTCACTGCCCGAGA
(SEQ ID NO: 59)
GGGAAGTCTTCGAGGTGCCC
(SEQ ID NO: 60)
GGTCAACATGTGCGTGGCTA
(SEQ ID NO: 61)
GGGCAGTGACGGCTCCCCTT
(SEQ ID NO: 62)
GTGACGGCTCCCCTTTGGCT
(SEQ ID NO: 63)
GTCGTGGGGGGAAGTCTTCG

In preferred embodiments, the PTP1B inhibitor is specific for PTP1B such that any off-target effects from the inhibitor are minimal. For example, preferably, the only protein that is inhibited by the PTP1B inhibitor, is PTP1B. Alternatively, the only phosphatase that is inhibited is PTP1B.

Moreover, it will be understood that any inhibitor selected for use in the methods of the present invention, is preferably an inhibitor that directly or specifically binds to or targets the activity or gene expression of PTPN1.

Preferably, the off-target effects of the inhibitor (for example an inhibitory RNA or CRISPR-based system) is such that a change in gene expression of any gene that is not PTPN1, is a reduction in gene expression of no more than about 5%, about 10%, about 20% or about 30%. Alternatively, the reduction in the expression of any gene that is not PTPN1, is a reduction of less than 30%, less than 20%, less than 10%, or less than 5%.

Preferably, the off-target effects of the inhibitor (for example a small molecule inhibitor, inhibitor peptide, antibody, preferably intrabody, or PROTAC) is such that the activity of any protein that is not PTP1B (or PTPN2), is a reduction in activity of no more than about 5%, about 10%, about 20% or about 30%. Alternatively, the reduction in the activity of any protein that is not PTP1B, is a reduction of less than 30%, less than 20%, less than 10%, or less than 5%.

PTPN2 and PTPN2 Inhibitors

PTPN2 (also known as T cell PTP, PTN2, PTPT, TC-PTP, TCELLPTP and TCPTP) is a ubiquitous phosphatase that is expressed abundantly in hematopoietic cells, including T cells. Two splice variants of TCPTP are expressed that have identical N termini and catalytic domains but varied C termini: a 48-kDa form (TC48) that is targeted to the endoplasmic reticulum (ER) by a hydrophobic C terminus and a 45-kDa variant (TC45) that is targeted to the nucleus by a nuclear localization sequence. Despite an apparently exclusive nuclear localization in resting cells, TC45 can shuttle between the nucleus and cytoplasm to access substrates in both compartments. Genome-wide association studies have linked PTPN2 single nucleotide polymorphisms (SNPs) with the development of several human autoimmune diseases including type 1 diabetes, rheumatoid arthritis, Crohn's disease and celiac disease. In particular, an intronic PTPN2 variant, rs1893217(C), has been linked with the development of type 1 diabetes. This SNP is associated with an approximate 40% decrease in PTPN2 mRNA in CD4+ T cells. PTPN2 is a key regulator of TCR signaling in naive CD4+ and CD8+ T cells and functions to dephosphorylate and inactivate Lck and Fyn. PTPN2 also dephosphorylates Janus-activated kinases (JAK)-1/3 and signal transducers and activator of transcription (STAT)-1/3/5/6 to attenuate cytokine signaling.

In order to determine if the presence of a PTPN2 inhibitor has inhibited PTPN2, experiments such as the following could be performed: measure PTPN2 activity in PTPN2 immunoprecipitates using p-NPP (para-nitrophenylphosphate) and p-tyr-RCML (p-tyr-reduced, carboxyamidomethylated and maleylated lysozyme) as substrates as described previously (Bukczynska P et al. Biochem J. 2004 Jun. 15; 380(Pt 3):939-49; Tiganis T et al. J Biol Chem. 1997 Aug. 22; 272(34):21548-57). Alternatively, analysis of known substrates of PTPN2 such as Src-family kinase members Lck and Fyn and transcription factors STAT1, STAT3 and STAT5 for tyrosine-phosphorylation by flow cytometry and immuno-blotting can be performed.

As used herein, a “compound that inhibits PTPN2”, or an “PTPN2 inhibitor” or “inhibitor of PTPN2” is any compound that inhibits the activity of PTPN2, for example, completely or partially reduces one or more functions of PTPN2 including those as described herein. Inhibition of activity of PTPN2 may also include a reduction in the level or amount of PTPN2 protein, RNA or DNA in a cell. The compound may be a competitive, non-competitive, orthosteric, allosteric, or partial inhibitor. In a preferred form the compound is a molecule that inhibits the enzyme activity.

A PTPN2 inhibitor useful in the present invention is one that completely or partially reduces one or more functions of PTPN2 as described herein. Preferably, a PTPN2 inhibitor reduces phosphatase activity of PTPN2 (such as a small molecule, peptide or peptidomimetic, antibody, preferably intrabody, or PROTAC), reduces the transcriptional activity of the Ptpn2 gene, or reduces the amount of PTPN2 mRNA or protein present in the cell.

In any embodiment of the invention, the inhibition of PTPN2 may be inhibition of at least 50%, at least 60%, at least 70%, at least 80%, at least 90% or 100% inhibition. In further embodiments, the inhibition is of PTPN2 only, such that there are minimal-to-no off-target effects resulting in inhibition of other targets. Accordingly, in preferred embodiments, the inhibition of targets other than PTPN2 is no more than 20%, no more than 10%, no more than 5% inhibition.

As used herein, a PTPN2 inhibitor may be any molecule that inhibits the phosphatase activity of PTPN2 or reduces the level of PTPN2 in a cell. The inhibitor may be a direct inhibitor of the phosphatase active site, may act allosterically to inhibit phosphatase activity, inhibit interaction of PTPN2 with its substrate, or may reduce the level of PTPN2 by reducing the transcriptional activity of the Ptpn2 gene, or reducing the amount of PTPN2 mRNA or protein present in the cell.

Exemplary small molecules that inhibit PTPN2 and that are useful in the present invention are ethyl-3,4-dephospatin or compound 8 (Zhang et al. (2009), JACS, 131, 13072 to 13079). Other inhibitors that may be useful in the invention include molecules with PTPN2 inhibitory activity as described in WO03/073987 A2; WO 03/097621 A1; US 2012/0088720 A1; U.S. Pat. No. 7,393,869; and US 2006/0235061 A1.

Chemical Structure of Ethyl-3,4-Dephospatin:

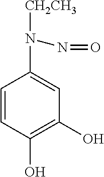 [SMILES: CCN(N=O)c1ccc(O)c(O)c1]

Chemical Structure of Compound 8:

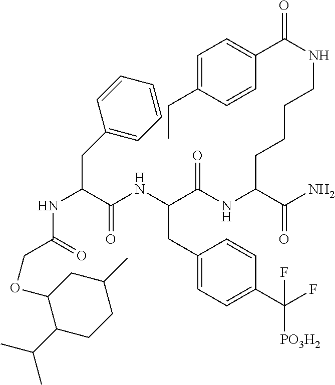

As used herein, reference to PTPN2 inhibitor or inhibitor of PTPN2 also includes a pharmaceutically acceptable salt, ester, polymorph or prodrug thereof.

The inhibitor may also be a peptide, or peptidomimetic, or an antibody such as a monoclonal antibody, preferably wherein the antibody is an intrabody for inhibiting PTPN2.

In further embodiments, the inhibitor may be a PROTAC which targets PTPN2 for degradation.

Methods for generating PROTACs, including small-molecule, peptide-based PROTACs and PROTAC-antibody conjugates are known in the art (see for example, GB 2554071, WO 2018051107, WO 2016146985, WO2017/201449 and Zou et al., (2019), Cell Biochem Funct, 37: 21-30).

The expression of PTPN2 can be reduced by any means that reduces the level of PTPN2 transcription. For example, miRNA, shRNA or siRNA approaches can be used.

Exemplary siRNA and shRNA include any one or more of the following sequences or sequences having sufficient homology to reduce expression of PTPN2 by targeting the coding sequence of PTPN2 or the 3′UTR.

Exemplary siRNA includes:

SEQ ID NO: 64
(5=AAGAUUGACAGACACCUAAUAUU3=);
and
SEQ ID NO: 65
(5=AAGCCCAUAUGAUCACAGUCG3=);

and exemplary shRNA include:

TRCN0000002781, with a target sequence of GATGACCAAGAGATGCTGTTT (SEQ ID NO: 93) beginning at position 582 of PTPN2 sequence from NM_001207013.1 and a hairpin sequence of:

SEQ ID NO: 66;
5′-CCGG-GATGACCAAGAGATGCTGTTT-CTCGAG-
AAACAGCATCTCTTGGTCATC-TTTTT-3′;

TRCN0000002782, with a target sequence of TGCAAGATACAATGGAGGAGA (SEQ ID NO: 94) beginning at position 1273 of PTPN2 sequence from NM_001207013.1 and a hairpin sequence of:

SEQ ID NO: 67;
5′-CCGG-TGCAAGATACAATGGAGGAGA-CTCGAG-
TCTCCTCCATTGTATCTTGCA-TTTTT-3′;

TRCN0000002783, with a target sequence of GAAGATGTGAAGTCGTATTAT (SEQ ID NO: 95) beginning at position 636 of PTPN2 sequence from NM_001207013.1 and a hairpin sequence of:

SEQ ID NO: 68;
5′-CCGG-GAAGATGTGAAGTCGTATTAT-CTCGAG-
ATAATACGACTTCACATCTTC-TTTTT-3′;

TRCN0000002784, with a target sequence of GTGCAGTAGAATAGACATCAA (SEQ ID NO: 96) beginning at position 1542 of PTPN2 sequence from NM_002828.3 and a hairpin sequence of:

SEQ ID NO: 69;
5′-CCGG-GTGCAGTAGAATAGACATCAA-CTCGAG-
TTGATGTCTATTCTACTGCAC-TTTTT-3′;

TRCN0000002785, with a target sequence of CTCACTTTCATTATACTACCT (SEQ ID NO: 97) beginning at position 781 of PTPN2 sequence from NM_001207013.1 and a hairpin sequence of:

SEQ ID NO: 70;
5′-CCGG-CTCACTTTCATTATACTACCT-CTCGAG-
AGGTAGTATAATGAAAGTGAG-TTTTT-3′;

TRCN0000314692, with a target sequence of ATTCTCATACATGGCTATAAT (SEQ ID NO: 98) beginning at position 1061 of PTPN2 sequence from NM_001207013.1 and a hairpin sequence of:

SEQ ID NO: 71;
5′-CCGG-ATTCTCATACATGGCTATAAT-CTCGAG-
ATTATAGCCATGTATGAGAAT-TTTTTG-3′;

TRCN0000314609, with a target sequence of AGAAGATGTGAAGTCGTATTA (SEQ ID NO: 99) beginning at position 635 of PTPN2 sequence from NM_001207013.1 and a hairpin sequence of:

SEQ ID NO: 72;
5′-CCGG-AGAAGATGTGAAGTCGTATTA-CTCGAG-
TAATACGACTTCACATCTTCT-TTTTTG-3′;

TRCN0000279329, with a target sequence of ATATGATCACAGTCGTGTTAA (SEQ ID NO: 100) beginning at position 270 of PTPN2 sequence from NM_001127177.1 and a hairpin sequence of:

SEQ ID NO: 73;
5′-CCGG-ATATGATCACAGTCGTGTTAA-CTCGAG-
TTAACACGACTGTGATCATAT-TTTTTG-3′;

TRCN0000314612, with a target sequence of GTGGAGAAAGAATCGGTTAAA (SEQ ID NO: 101) beginning at position 540 of PTPN2 sequence from NM_001207013.1 and a hairpin sequence of:

SEQ ID NO: 74;
5′-CCGG-GTGGAGAAAGAATCGGTTAAA-CTCGAG-
TTTAACCGATTCTTTCTCCAC-TTTTTG-3′;

TRCN0000314693, with a target sequence of TATGATCACAGTCGTGTTAAA (SEQ ID NO: 102) beginning at position 354 of PTPN2 sequence from NM_001207013.1 and a hairpin sequence of:

SEQ ID NO: 75;
5′-CCGG-TATGATCACAGTCGTGTTAAA-CTCGAG-
TTTAACACGACTGTGATCATA-TTTTTG-3′;

TRCN0000029891, with a target sequence of GCCAAGATTGACAGACACCTA (SEQ ID NO: 103) beginning at position 8031 of PTPN2 sequence from NM_001127177.1 and a hairpin sequence of:

SEQ ID NO: 76;
5′-CCGG-GCCAAGATTGACAGACACCTA-CTCGAG-
TAGGTGTCTGTCAATCTTGGC-TTTTT-3′;

TRCN0000314551, with a target sequence of GTGCAGTAGAATAGACATCAA (SEQ ID NO: 104) beginning at position 1542 of PTPN2 sequence from NM_002828.3 and a hairpin sequence of:

SEQ ID NO: 77.
5′-CCGG-GTGCAGTAGAATAGACATCAA-CTCGAG-
TTGATGTCTATTCTACTGCAC-TTTTTG-3′:

Further, the inhibition of PTPN2 may also include genome editing to delete or modify all or part of a sequence encoding PTPN2. The genome editing may be a modification that includes an insertion, deletion, integration of sequence modification/substitution such that the expression of functional PTPN2 protein is reduced or ablated. Genome editing techniques are well known in the art and include the use of various nucleases including TALENs, zinc finger nucleases and meganucleases.

The inhibitor may therefore be in the form of a compound/molecule for use in genome editing to remove or modify all or part of a sequence encoding PTPN2. In one example, the genome-editing molecule may be a TALEN, meganuclease or a zinc-finger nuclease which is specifically designed to remove or modify all or part of a sequence encoding PTPN2.

Another exemplary genome editing technique is the CRISPR/Cas9 system (Jinek, M., et al. (2012) Science, 337, 816-821; Cong L., et al. (2013) Science, 339, 819-823; and Qi, L. S., et al. (2013) Cell, 152, 1173-1183) and related technology including but not limited to CRISPR/Cas12a and CRISPR/Cas13. As such, in accordance with the present invention, the PTPN2 inhibitor may include a gRNA (including an sgRNA) for use in CRISPR genome editing to inhibit or delete PTPN2 activity. More specifically, the present invention contemplates the use of CRISPR-Cas9 to delete Ptpn2 in human CAR T cells. Moreover, use of CRISPR-Cas9 enables the inhibition to be of PTPN2 alone (i.e., wherein only PTPN2 is inhibited, or where there are minimal off-target effects). In certain embodiments, the inhibition of only PTPN2 may be complete inhibition (i.e., knock-out) of PTPN2 function, or a reduction in PTPN2 activity/expression (i.e., knock-down or partial knock-out).

The skilled person will be able to purchase or design gRNAs or crRNAs which target a variety of PTPN2 sequences. Examples of such gRNA target sequences include:

(SEQ ID NO: 78)
CTCTTCGAACTCCCGCTCGA
(SEQ ID NO: 79)
AGTTGGATACTCAGCGTCGC
(SEQ ID NO: 80)
CCATGACTATCCTCATAGAG
(SEQ ID NO: 81)
CCACTCTATGAGGATAGTCA
(SEQ ID NO: 82)
CTCTTCTATGTCAACTAAAC
(SEQ ID NO: 83)
CAGTTTAGTTGACATAGAAG
(SEQ ID NO: 84)
TTCGAACTCCCGCTCGATGG
(SEQ ID NO: 85)
CCATGACTATCCTCATAGAG
(SEQ ID NO: 86)
TTGACATAGAAGAGGCACAA

In preferred embodiments, the PTPN2 inhibitor is specific for PTPN2 such that any off-target effects from the inhibitor are minimal. For example, preferably, the only protein that is inhibited is PTPN2. Alternatively, the only phosphatase that is inhibited is PTPN2.

Moreover, it will be understood that any inhibitor selected for use in the methods of the present invention, is preferably an inhibitor that directly or specifically binds to or targets the activity or gene expression of PTPN2.

Preferably, the off-target effects of the inhibitor (for example an inhibitory RNA or CRISPR-based system) is such that a change in gene expression of any gene that is not Ptpn2, is a reduction in gene expression of no more than about 5%, about 10%, about 20% or about 30%. Alternatively, the reduction in the expression of any gene that is not Ptpn2, is a reduction of less than 30%, less than 20%, less than 10%, or less than 5%.

Preferably, the off-target effects of the PTPN2 inhibitor (for example a small molecule inhibitor, inhibitor peptide, antibody, preferably intrabody or PROTAC) is such that the activity of any protein that is not PTPN2, is a reduction in activity of no more than about 5%, about 10%, about 20% or about 30%. Alternatively, the reduction in the activity of any protein that is not PTPN2, is a reduction of less than 30%, less than 20%, less than 10%, or less than 5%.

Methods for Modifying Cells

ZFNs are artificial restriction enzymes generated by fusing a zinc finger DNA-binding domain to a DNA-cleavage domain. Zinc finger domains can be engineered to target desired DNA sequences, which enables zinc-finger nucleases to target a unique sequence within a complex genome. By taking advantage of endogenous DNA repair machinery, these reagents can be used to precisely alter the genomes of higher organisms. Other technologies for genome customization that can be used to knock out genes are meganucleases and TAL effector nucleases (TALENs, Cellectis bioresearch). A TALEN® is composed of a TALE DNA binding domain for sequence-specific recognition fused to the catalytic domain of an endonuclease that introduces double strand breaks (DSB). The DNA binding domain of a TALEN® is capable of targeting with high precision a large recognition site (for instance 17 bp). Meganucleases are sequence-specific endonucleases, naturally occurring “DNA scissors”, originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes). The skilled person will be familiar with standard methods for generating such TALENs, meganucleases or zinc-finger nucleases (ZFNs). Exemplary methods are described, for example in: Gaj et al., (2013) Trends Biotechnol, 31:397-405.

In any embodiment of the present invention, the miRNA, siRNA or shRNA inhibitor (whether of PTPN2 and/or PTP1B) can be delivered to the relevant cell (including a CAR T cell) by using a viral vector. There are a large number of available viral vectors that are suitable for use with the present invention, including those identified for human gene therapy applications. Suitable viral vectors include vectors based on RNA viruses, such as retrovirus-derived vectors, e.g., Moloney murine leukemia virus (MLV)-derived vectors, and include more complex retrovirus-derived vectors, e.g., Lentivirus-derived vectors. Human Immunodeficiency virus (HIN-I)-derived vectors belong to this category. Other examples include lentivirus vectors derived from HIN-2, feline immunodeficiency virus (FIN), equine infectious anemia virus, simian immunodeficiency virus (SIV) and Maedi-Visna virus.

Preferably a modified retrovirus, even more preferably a modified lentivirus, is used to deliver the specific miRNA, siRNA or shRNA. This virus may also include sequences that encode the chimeric antigen receptor for targeting the specific cell to be killed. The polynucleotide and any associated genetic elements are thus integrated into the genome of the host cell as a provirus. The modified retrovirus is preferably produced in a packaging cell from a viral vector that includes the sequences necessary for production of the virus as well as the miRNA, siRNA or shRNA and/or CAR. The viral vector may also include genetic elements that facilitate expression of the miRNA, siRNA or shRNA, such as promoter and enhancer sequences. In order to prevent replication in the target cell, endogenous viral genes required for replication may be removed.

The skilled person will also be familiar with methods for virally introducing Cas9 and guide RNAs (gRNAs) into cells for the purpose of targeting PTP1B and/or PTPN2 (for example, utilising lentiviral methods). In addition, the present invention contemplates the use of Cas9 ribonucleoprotein (RNP)-mediated gene-editing to delete PTP1B and/or PTPN2 (for example using GeneArt™ Platinum™ Cas9 Nuclease pre-loaded with synthesized crRNA:tracrRNA (Dharmacon) targeting human PTP1B or PTPN2 using the Neon Transfection system).

In further embodiments, Cas9 and gRNA, or indeed any inhibitory RNA molecule, may be introduced into T cells using non-viral methods, including electroporation. For example, the sgRNAs and Cas9 may be introduced into T cells (including CAR T cells) using the T cell Nucleofector system (Lonza Bioscience).

The skilled person will be able to determine whether PTP1B and/or PTPN2 mRNA levels have been reduced using standard quantitative PCR methods. For example, the Taqman gene expression assay to determine Ptpn1 and Ptpn2 expression can be used (Mm00448427_m1, and Hs00741253_m1, respectively, Thermofisher Scientific). The skilled person will understand that such assays can be used to confirm PTP1B mRNA and/or PTPN2 mRNA reduction resulting from siRNA or shRNA targeting or alternatively as the result of gRNA-derived CRISPR-Cas9 genome editing to reduce PTP1B and/or PTPN2 activity.

Deletion of PTPN1 and/or PTPN2 can also be determined using standard flow cytometry techniques, as further described herein in the Examples.

A composition comprising the cytotoxic leukocytes (e.g., CD8+ T cells, NK cells, etc), a PTP1B inhibitor and/or a PTPN2 inhibitor as described herein, may further include the cancer specific antigen and/or one or more cytokines to enhance cell killing (such as IL-2 or IFNγ). When the antigen is present in the composition comprising the isolated, enriched or purified cytotoxic leukocytes, the antigen may be present as an independent entity, or in any context by which the antigen can interact with a receptor or CAR present on the cells. When the antigen can interact with the TCR of the CD8+ T cells the CD8+ T cells can become activated. Examples of various embodiments by which the antigen can be provided in the composition such that it can be recognized by the CD8+ TCR include but are not limited to it the antigen being present in association with MHC-I (or the equivalent presentation in an animal model) on the surface of antigen presenting cells, such as dendritic cells, macrophages or certain activated epithelial cells. Alternatively, the antigen could be in physical association with any other natural or synthesized molecule or other compound, complex, entity, substrate, etc., that would facilitate the recognition of the antigen by the TCR on the CD8+ T cells. For example, the antigen could be complexed to a MHC-I or other suitable molecule for presenting the antigen to the CD8+ TCR, and the MHC-I or other suitable molecule could be in physical association with a substrate, such as a latex bead, plastic surface of any plate, or any other suitable substrate, to facilitate appropriate access of the antigen to the CD8+ T cell TCR such that the antigen is recognized by the CD8+ T cell.

CD8+ T cells may be obtained using routine cell sorting techniques that discriminate and segregate T cells based on T cell surface markers can be used to obtain an isolated population CD8+ T cells for use in the compositions and methods of the invention. For example, a biological sample including blood and/or peripheral blood lymphocytes can be obtained from an individual and CD8+ T cells isolated from the sample using commercially available devices and reagents, thereby obtaining an isolated population of CD8+ T cells. Murine CD8+ T cells may be further characterized and/or isolated on a phenotypic basis via the use of additional cell surface markers such as CD44, L-selectin (CD62L), CD25, CD49d, CD122, CD27, CD43, CD69, KLRG-1, CXCR3, CCR7, IL-7Rα and KLRG-1. CD8+ T cells may be initially enriched by negatively selecting CD4+, NK1.1+, B220+, CD11b+, TER119+, Gr-1+, CD11c+ and CD19+ cells. Naive CD8+ T cells are characterized as CD44 low, CD62L high, CCR7 high, CD25 low, CD43 low, CD49d low, CD69 low, IL-7Rα high and CD122 low, whereas antigen experienced memory T cells are CD44 high, CD49d high, CD122 high, CD27 high, CD43 high and CXCR3 high. Memory CD8+CD44 high T cells can be further sub-divided into lymphoid-tissue residing Central Memory T cells (CD62L high, CCR7 high) and non-lymphoid tissue residing Effector Memory T cells (CD62L low, CCR7 low) (Klonowski et al. Immunity 2004, 20:551-562). The isolated population of CD8+ T cells can be mixed with the PTP1B and/or antigen in any suitable container, device, cell culture media, system, etc., and can be cultured in vitro and/or exposed to the one or more antigens, and any other reagent, or cell culture media, in order to expand and/or mature and/or differentiate the T cells to have any of various desired cytotoxic T cell characteristics.

Human CD8+ T-cell types and/or populations can be identified using the phenotypic cell-surface markers CD62L, CCR7, CD27, CD28 and CD45RA or CD45RO (Sallusto F et al. Nature 1999, 401:708-712). As used herein, CD8+ T-cell types and/or populations have the following characteristics or pattern of expression of cell surface markers: Naive T cells are characterized as CD45RA+, CD27+, CD28+, CD62L+ and CCR7+; CD45RO−; Central Memory T cells are CD45RA−, CD27+, CD28+, CD62L+ and CCR7+; CD45RO+ Effector Memory T cells are defined by the lack of expression of these five markers (CD45RA−, CD27−, CD28−, CD62L− and CCR7−); and terminally differentiated Effector Memory T cells are characterized as CD45RO+, CCR7−, CD27−, CD28−, CD62L−. Terminally differentiated Effector Memory cells further up-regulate markers such as CD57, KLRG1, CX3CR1 and exhibit strong cytotoxic properties characterized by their ability to produce high levels of Granzyme A and B, Perforin and IFNγ. Therefore, various populations of T cells can be separated from other cells and/or from each other based on their expression or lack of expression of these markers. In this manner, the invention provides methods of separating different populations of CD8+T cells and also separated or isolated populations of CD8+ T cells. The CD8+ T cell types described herein may also be isolated by any other suitable method known in the art; for example, if a particular antigen or antigens are used to produce antigen-specific CD8+ T cells, those cells can be separated or isolated from other cells by affinity purification using that antigen or antigens; appropriate protocols are known in the art.

Different CD8+ T cell types can also exhibit particular functions, including, for example: secretion of IFN-γ; secretion of IL-2; production of Granzyme B; expression of FasL and expression of CD107. However, while the expression pattern of cell surface markers is considered diagnostic of each particular CD8+ T cell type and/or population as described herein, the functional attributes of each cell type and/or population may vary depending on the amount of stimulation the cell(s) has or have received.

Effector functions or properties of T cells can be determined by the effector molecules that they release in response to specific binding of their T-cell receptor with antigen:MHC complex on the target cell, or in the case of CAR T-cells interaction of the chimeric antigen receptor, e.g. scFv, with the antigen expressed on the target cell. Cytotoxic effector molecules that can be released by cytotoxic CD8+ T cells include perforin, granzymes A and B, granulysin and Fas ligand. Generally, upon degranulation, perforin inserts itself into the target cell's plasma membrane, forming a pore, granzymes are serine proteases which can trigger apoptosis (a form of cell death), granulysin induces apoptosis in target cells, and Fas ligand can also induce apoptosis. Typically, these cytotoxic effector molecules are stored in lytic granules in the cell prior to release. Other effector molecules that can be released by cytotoxic T cells include IFN-γ, LTα, TNF-β and TNF-α. IFN-γ can inhibit viral replication and activate macrophages, while LTα, TNF-β and TNF-α can participate in macrophage activation and in killing target cells. In any method of the invention, before administration or reintroduction of the cells contacted with a PTP1B inhibitor, those cells will be assessed for their cytotoxic activity by flow cytometry using fluorochrome-conjugated antibodies against surface and intracellular markers that specify cytotoxic effector T cells including Granzyme A and B, Perforin and IFNγ.

An activated T cell is a cell that is no longer in GO phase, and begins to produce one or more cytotoxins, cytokines and/or other membrane-associated markers characteristic of the cell type (e.g., CD8+) as described herein and is capable of recognizing and binding any target cell that displays the particular peptide:MHC complex or antigen alone on its surface and releasing its effector molecules.

The methods of the invention that promote the differentiation of T cells into a population of cytotoxic T cells lead to a statistically significant increase in the population of cytotoxic T cells. A population is increased when the cells are present in an amount which is at least 10%, 20%, 30%, 40%, 50%, 60%, 70%, 80%, 90%, 95%, or 100% higher in comparison to an appropriate control such as, for example, the size of the population prior to treatment with a method of the invention. The cytotoxic CD8+ T cell effector function is increased when cells have a function which is at least 10%, 20%, 30%, 40%, 50%, 60%, 70%, 80%, 90%, 95%, or 100% higher, than an appropriate control, such as, for example, the performance of a sample of cells in a particular assay in the absence of a particular event or condition. Where appropriate, in vivo function or the presence of a cell population in vivo may be measured using cells isolated from a subject in in vitro assays.

An “enriched” or “purified” population of cells is an increase in the ratio of particular cells to other cells, for example, in comparison to the cells as found in a subject's body, or in comparison to the ratio prior to exposure to a PTP1B inhibitor and/or a PTPN2 inhibitor. In some embodiments, in an enriched or purified population of cells, the particular cells include at least 20%, 30%, 40%, 50%, 60%, 70%, 75%, 80%, 90%, 95% or 99% of the total cell population. A population of cells may be defined by one or more cell surface markers and/or properties.

Cytotoxic leukocytes exposed to, or contacted with, a PTP1B inhibitor and/or a PTPN2 inhibitor that exhibit at least one property of a cytotoxic cell as described herein, upon administration to the subject, elicit a cytotoxic cell response to a tumour cell. Preferably, that response to a tumour cell is effective in causing cell death, such as lysis, of tumour cells having the targeted antigen.

Cytotoxic leukocytes exposed to, or contacted with, a PTP1B inhibitor and/or a PTPN2 inhibitor can be administered to the subject by any method including, for example, injection, infusion, deposition, implantation, oral ingestion, or topical administration, or any combination thereof. Injections can be, e.g., intravenous, intramuscular, intradermal, subcutaneous or intraperitoneal. Single or multiple doses can be administered over a given time period, depending upon the cancer, the severity thereof and the overall health of the subject, as can be determined by one skilled in the art without undue experimentation. The injections can be given at multiple locations. Administration of the cytotoxic leukocytes can be alone or in combination with other therapeutic agents. Each dose can include about 10×10³ cytotoxic leukocytes, 20×10³ cells, 50×10³ cells, 100×10³ cells, 200×10³ cells, 500×10³ cells, 1×10⁶ cells, 2×10⁶ cells, 20×10⁶ cells, 50×10⁶ cells, 100×10⁶ cells, 200×10⁶, 500×10⁶, 1×10⁹ cells, 2×10⁹ cells, 5×10⁹ cells, 10×10⁹ cells, and the like. Administration frequency can be, for example, once per week, twice per week, once every two weeks, once every three weeks, once every four weeks, once per month, once every two months, once every three months, once every four months, once every five months, once every six months, and so on. The total number of days where administration occurs can be one day, on 2 days, or on 3, 4, 5, 6, 7, 8, 9, 10, 11, 12, 13, 14, 15, 16, 17, 18, 19, or 20 days, and so on. It is understood that any given administration might involve two or more injections on the same day. For administration, at least 20%, at least 30%, at least 40%, at least 50%, at least 60%, at least 70%, at least 80%, at least 90%, at least 95%, at least 99%, of the cytotoxic leukocytes that are administered exhibit at least one property of a cytotoxic cell.

In one illustrative embodiment, when the cells have been treated with a PTP1B inhibitor and/or a PTPN2 (such as a small molecule inhibitor, an inhibitory RNA or including an inhibitor in the form of CRISPR/Cas9 system for inhibiting PTP1B and/or PTPN2), a composition comprising the cytotoxic leukocytes can be prepared and administered to the patient.

It will be understood that in accordance with the methods of the present invention, the mode of inhibition of PTP1B and of PTPN2 does not need to be the same. For example, the methods encompass scenarios wherein PTP1B is inhibited directly in the cells to be administered to a subject (for example, by treating leukocytes proposed to be administered to the subject, with a PTP1B inhibitor, ex vivo), and the PTPN2 inhibitor is administered directly to the subject before, at the same time, sequentially or after the PTP1B-inhibited cells are administered. Conversely, when PTPN2 is inhibited directly in the cells to be administered to a subject (for example, by treating leukocytes proposed to be administered to the subject, with a PTPN2 inhibitor, ex vivo), the PTP1B inhibitor may be administered directly to the subject before, at the same time, sequentially or after the PTPN2-inhibited cells are administered. In a further alternative, both PTP1B and PTPN2 inhibitors may be administered directly to a subject who is about to, or has received immunotherapy with T cells, or both PTP1B and PTPN2 may be inhibited in cells that are proposed to be administered to a subject for the purposes of immunotherapy.

In one embodiment, culture media that lacks any animal products, such as bovine serum, can be used to culture the cytotoxic leukocytes. In another embodiment, tissue culture conditions typically used by the skilled artisan to avoid contamination with bacteria, fungi and mycoplasma can be used. In an exemplary embodiment, prior to being administered to a patient, the cytotoxic leukocytes (e.g. CAR T cells or CAR NK cells) are pelleted, washed, and are resuspended in a pharmaceutically acceptable carrier or diluent. Exemplary compositions comprising CAR-expressing T lymphocytes (e.g., cytotoxic T lymphocytes) include compositions comprising the cells in sterile 290 mOsm saline, in infusible cryomedia (containing Plasma-Lyte A, dextrose, sodium chloride injection, human serum albumin and DMSO), in 0.9% NaCl with 2% human serum albumin, or in any other sterile 290 mOsm infusible materials. Alternatively, in another embodiment, depending on the identity of the culture medium, the CAR-T cells can be administered in the culture media as the composition, or concentrated and resuspended in the culture medium before administration. In various embodiments, the CAR-T cell composition, can be administered to the patient via any suitable means, such as parenteral administration, e.g., intradermally, subcutaneously, intramuscularly, intraperitoneally, intravenously, or intrathecally.

Administration and Indications to be Treated

In further embodiments, the present application includes administration of a PTP1B inhibitor and/or a PTPN2 inhibitor directly to an individual who is receiving or has received a treatment with cytotoxic leukocytes. The cytotoxic leukocytes may have been contacted with a PTP1B inhibitor and/or PTPN2 prior to administration to a subject requiring treatment, according to any method described herein. Alternatively, the cytotoxic leukocytes are administered to the subject, without receiving prior exposure or contact with a PTP1B inhibitor, and instead, the PTP1B inhibitor is administered directly to the subject. In this embodiment, the cytotoxic leukocytes may have received prior exposure of contact with a PTPN2 inhibitor.

Alternatively, the cytotoxic leukocytes are administered to the subject, without receiving prior exposure or contact with a PTPN2 inhibitor, and instead, the PTPN2 inhibitor is administered directly to the subject. In this embodiment, the cytotoxic leukocytes may have received prior exposure or contact with a PTP1B inhibitor.

The PTP1B inhibitor and/or PTPN2 inhibitor may be administered prior to, at the same time as, or after the subject receives treatment with the cytotoxic leukocyte. Where the PTP1B inhibitor and/or PTPN2 inhibitor and cytotoxic leukocytes are administered to the subject at the same time, they can be administered via the same route of administration (including in a single composition), or alternatively via different routes of administration. For example, the cytotoxic leukocytes may be administered by injection into the blood stream of the subject, while the PTP1B and/or PTPN2 inhibitor may be administered orally, or via another route of administration such as intramuscularly, intradermally, subcutaneously or intraperitoneally.

In one preferred embodiment, the PTP1B inhibitor and/or PTPN2 inhibitor is directly administered to the subject following administration of CAR T cells to the subject, for the purpose of enhancing the efficacy of the CAR T treatment. The inhibitor can be subsequently administered once every two weeks, or once or twice weekly, or more, to facilitate CAR T cell expansion and the formation of memory CAR T cells.

In certain embodiments, the PTP1B inhibitor is trodusquemine, administered by injection, or a derivative (for example DPM-1001) administered orally before, during or after intravenous administration of CAR T cells.

In particularly preferred embodiments, a CAR T cell is genetically modified to delete part or all of the gene encoding PTPN2 (e.g., using a CRISPR-based system or any other genome editing technique known to the skilled person), and administered to a subject requiring treatment, wherein the treatment includes concomitant or subsequent administration of a pharmacological PTP1B inhibitor, preferably trodusquemine (MSI-1436).

It will be clearly understood that, although this specification refers specifically to applications in humans, the invention is also useful for veterinary purposes. Thus in all aspects the invention is useful for domestic animals such as cattle, sheep, horses and poultry; for companion animals such as cats and dogs; and for zoo animals. Therefore, the general term “subject” or “subject to be/being treated” is understood to include all animals (such as humans, apes, dogs, cats, horses, and cows) that require an enhanced immune response, for example subjects having cancer.

As used herein, the term “ex vivo” or “ex vivo therapy” refers to a therapy where cells are obtained from a patient or a suitable alternate source, such as, a suitable allogenic donor, and are modified, such that the modified cells can be used to treat a disease which will be improved by the therapeutic benefit produced by the modified cells. Treatment includes the administration or re-introduction of the modified cells into the patient. A benefit of ex vivo therapy is the ability to provide the patient the benefit of the treatment, without exposing the patient to undesired collateral effects from the treatment.

The term “administered” means administration of a therapeutically effective dose of the aforementioned composition including the respective cells to an individual. By “therapeutically effective amount” is meant a dose that produces the effects for which it is administered. The exact dose will depend on the purpose of the treatment, and will be ascertainable by one skilled in the art using known techniques. As is known in the art and described above, adjustments for systemic versus localized delivery, age, body weight, general health, sex, diet, time of administration, drug interaction and the severity of the condition may be necessary, and will be ascertainable with routine experimentation by those skilled in the art.

Subjects requiring treatment include those already having a benign, pre-cancerous, or non-metastatic tumour as well as those in which the occurrence or recurrence of cancer is to be prevented. Subjects may have metastatic cells, including metastatic cells present in the ascites fluid and/or lymph node.

The objective or outcome of treatment may be to reduce the number of cancer cells; reduce the primary tumour size; inhibit (i.e., slow to some extent and preferably stop) cancer cell infiltration into peripheral organs; inhibit (i.e., slow to some extent and preferably stop) tumour metastasis; inhibit, to some extent, tumour growth; and/or relieve to some extent one or more of the symptoms associated with the disorder.

Efficacy of treatment can be measured by assessing the duration of survival, time to disease progression, the response rates (RR), duration of response, and/or quality of life.

The method is particularly useful for extending time to disease progression.

The method is particularly useful for extending survival of the human, including overall survival as well as progression free survival.

The method is particularly useful for providing a complete response to therapy whereby all signs of cancer in response to treatment have disappeared. This does not always mean the cancer has been cured.

The method is particularly useful for providing a partial response to therapy whereby there has been a decrease in the size of one or more tumours or lesions, or in the extent of cancer in the body, in response to treatment.

The objective or outcome of treatment may be any one or more of the following:
to reduce the number of cancer cells;
reduce the primary tumour size;
inhibit (i.e., slow to some extent and preferably stop) cancer cell infiltration into peripheral organs;
inhibit (i.e., slow to some extent and preferably stop) tumour metastasis;
inhibit, to some extent, tumour growth;
relieve to some extent one or more of the symptoms associated with the disorder.

In one embodiment, animals requiring treatment include those having a benign, pre-cancerous, non-metastatic tumour.

In one embodiment, the cancer is pre-cancerous or pre-neoplastic.

In one embodiment, the cancer is a secondary cancer or metastases. The secondary cancer may be located in any organ or tissue, and particularly those organs or tissues having relatively higher hemodynamic pressures, such as lung, liver, kidney, pancreas, bowel and brain. The secondary cancer may be detected in the ascites fluid and/or lymph nodes.

In one embodiment, the cancer may be substantially undetectable.

“Pre-cancerous” or “pre-neoplasia” generally refers to a condition or a growth that typically precedes or develops into a cancer. A “pre-cancerous” growth may have cells that are characterized by abnormal cell cycle regulation, proliferation, or differentiation, which can be determined by markers of cell cycle.

In one embodiment, the cancer expresses the cell surface tumour antigen Her-2. An example of a cancer that expresses the cell surface tumour antigen Her-2 is a sarcoma.

In one embodiment, the cancer expresses the cell surface tumour antigen Lewis Y antigen. An example of a cancer that expresses the cell surface tumour antigen Lewis Y is acute myeloid leukaemia.

The cancer may be a solid or a “liquid” tumour. In other words, the cancer may be growth in a tissue (carcinoma, sarcoma, adenomas etc) or it may be a cancer present in bodily fluid such as in blood or bone marrow (e.g., lymphomas and leukaemias).

Other examples of cancer include blastoma (including medulloblastoma and retinoblastoma), sarcoma (including liposarcoma and synovial cell sarcoma), neuroendocrine tumours (including carcinoid tumours, gastrinoma, and islet cell cancer), mesothelioma, schwannoma (including acoustic neuroma), meningioma, adenocarcinoma, melanoma, leukemia or lymphoid malignancies, lung cancer including small-cell lung cancer (SCLC), non-small cell lung cancer (NSCLC), adenocarcinoma of the lung and squamous carcinoma of the lung, epidermoid lung cancer, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, tumours of the biliary tract, as well as head and neck cancer.

Pre-neoplastic, neoplastic and metastatic diseases are particular examples to which the methods of the invention may be applied. Broad examples include breast tumours, colorectal tumours, adenocarcinomas, mesothelioma, bladder tumours, prostate tumours, germ cell tumour, hepatoma/cholangio, carcinoma, neuroendocrine tumours, pituitary neoplasm, small round cell tumour, squamous cell cancer, melanoma, atypical fibroxanthoma, seminomas, nonseminomas, stromal leydig cell tumours, Sertoli cell tumours, skin tumours, kidney tumours, testicular tumours, brain tumours, ovarian tumours, stomach tumours, oral tumours, bladder tumours, bone tumours, cervical tumours, esophageal tumours, laryngeal tumours, liver tumours, lung tumours, vaginal tumours and Wilms' tumour.

Examples of particular cancers include but are not limited to adenocarcinoma, adenoma, adenofibroma, adenolymphoma, adontoma, AIDS related cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, ameloblastoma, angiokeratoma, angiolymphoid hyperplasia with eosinophilia, angioma sclerosing, angiomatosis, apudoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumours, breast cancer, branchioma, CNS tumours, carcinoid tumours, cervical cancer, childhood brain tumours, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, carcinoma (e.g. Walker, basal cell, basosquamous, Brown-Pearce, ductal, Ehrlich tumour, Krebs 2, Merkel cell, mucinous, non-small cell lung, oat cell, papillary, scirrhous, bronchiolar, bronchogenic, squamous cell, and transitional cell), carcinosarcoma, cervical dysplasia, cystosarcoma phyllodes, cementoma, chordoma, choristoma, chondrosarcoma, chondroblastoma, craniopharyngioma, cholangioma, cholesteatoma, cylindroma, cystadenocarcinoma, cystadenoma, dermatofibrosarcoma-protuberans, desmoplastic-small-round-cell-tumour, ductal carcinoma, dysgerminoam, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibroma, fibrosarcoma, gall bladder cancer, gastric cancer, gastrointestinal cancers, gastrointestinal-carcinoid-tumour, genitourinary cancers, germ cell tumours, gestationaltrophoblastic-disease, glioma, gynaecological cancers, giant cell tumours, ganglioneuroma, glioma, glomangioma, granulosa cell tumour, gynandroblastoma, haematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, hamartoma, hemangioendothelioma, hemangioma, hemangiopericytoma, hemangiosarcoma, hemangiosarcoma, histiocytic disorders, histiocytosis malignant, histiocytoma, hepatoma, hidradenoma, hondrosarcoma, immunoproliferative small, opoma, ontraocular melanoma, islet cell cancer, Kaposi's sarcoma, kidney cancer, langerhan's cell-histiocytosis, laryngeal cancer, leiomyosarcoma, leukemia, li-fraumeni syndrome, lip cancer, liposarcoma, liver cancer, lung cancer, lymphedema, lymphoma, Hodgkin's lymphoma, non-Hodgkin's lymphoma, leiomyosarcoma, leukemia (e.g. B-cell, mixed cell, null-cell, T-cell, T-cell chronic, HTLV-II associated, lymphangiosarcoma, lymphocytic acute, lymphocytic chronic, mast-cell and myeloid), leukosarcoma, leydig cell tumour, leiomyoma, lymphangioma, lymphangiocytoma, lymphangioma, lymphangiomyoma, lymphangiosarcoma, male breast cancer, malignant-rhabdoid-tumour-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, malignant carcinoid syndrome carcinoid heart disease, meningioma, melanoma, mesenchymoma, mesonephroma, myoblastoma, myoma, myosarcoma, myxoma, myxosarcoma, nasal cancer, nasopharyngeal cancer, nephroblastoma, neuroblastoma, neurofibromatosis, Nijmegen breakage syndrome, non-melanoma skin cancer, non-small-cell-lung-cancer-(nsclc), neurilemmoma, neuroblastoma, neuroepithelioma, neurofibromatosis, neurofibroma, neuroma, neoplasms (e.g. bone, breast, digestive system, colorectal, liver), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumours, pituitary cancer, polycythemia vera, prostate cancer, osteoma, osteosarcoma, ovarian carcinoma, papilloma, paraganglioma, paraganglioma nonchromaffin, pinealoma, plasmacytoma, protooncogene, rare-cancers-and-associated-disorders, renal cell carcinoma, retinoblastoma, rhabdomyosarcoma, Rothmund-Thomson syndrome, reticuloendotheliosis, rhabdomyoma, salivary gland cancer, sarcoma, schwannoma, Sezary syndrome, skin cancer, small cell lung cancer (sclc), small intestine cancer, soft tissue sarcoma, spinal cord tumours, squamous-cell-carcinoma-(skin), stomach cancer, synovial sarcoma, sarcoma (e.g. Ewing's experimental, Kaposi's and mast-cell sarcomas), Sertoli cell tumour, synovioma, testicular cancer, thymus cancer, thyroid cancer, transitional-cell-cancer-(bladder), transitional-cell-cancer-(renal-pelvis-/-ureter), trophoblastic cancer, teratoma, theca cell tumour, thymoma, trophoblastic tumour, urethral cancer, urinary system cancer, uroplakins, uterine sarcoma, uterus cancer, vaginal cancer, vulva cancer, Waldenstrom's macroglobulinemia and Wilms' tumour.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1: PTP1B-Deficiency Increases the Survival an Expansion of Adoptively Transferred Tumour-Specific T Cells

Ptpn1^{fl/fl} mice have previously been described previously (Bence et al., 2006 Nature Medicine 12, 917-24). To delete PTP1B in T cells, Ptpn1^{fl/fl} mice were crossed with Lck-Cre transgenic mice to generate Lck-Cre;Ptpn1^{fl/fl} mice. To generate OT-1;Lck-Cre;Ptpn1^{fl/fl} bearing a TCR specific for the ovalbumin (OVA) peptide SIINFEKL Lck-Cre;Ptpn1^{fl/fl} mice were crossed with OT-1 (C57BL/6-Tg(TcraTcrb)1100Mjb/Crl) mice (The Jackson Laboratory).

AT-3-OVA mammary tumour cells (1×10⁶) were injected into the fourth inguinal mammary fat pads of female Ly5.1⁺ mice. Seven days after tumour injection FACS-purified 2×10⁶ naïve CD8⁺CD44^{lo}CD62L^{hi} lymph node T cells from Ly5.2⁺;OT-1;Ptpn1^{fl/fl} versus Ly5.2⁺;OT-1;Lck-Cre;Ptpn1^{fl/fl} mice were adoptively transferred into tumour-bearing Ly5.1 mice. After 25 days T cells isolated from tumours, draining lymph nodes and spleen were processed for flow cytometry and donor T cell numbers (Ly5.1⁻Ly5.2⁺) and intracellular levels of the pro-survival protein Bcl-2 were assessed; Bcl-2 mean fluorescence intensities (MFI) were determined.

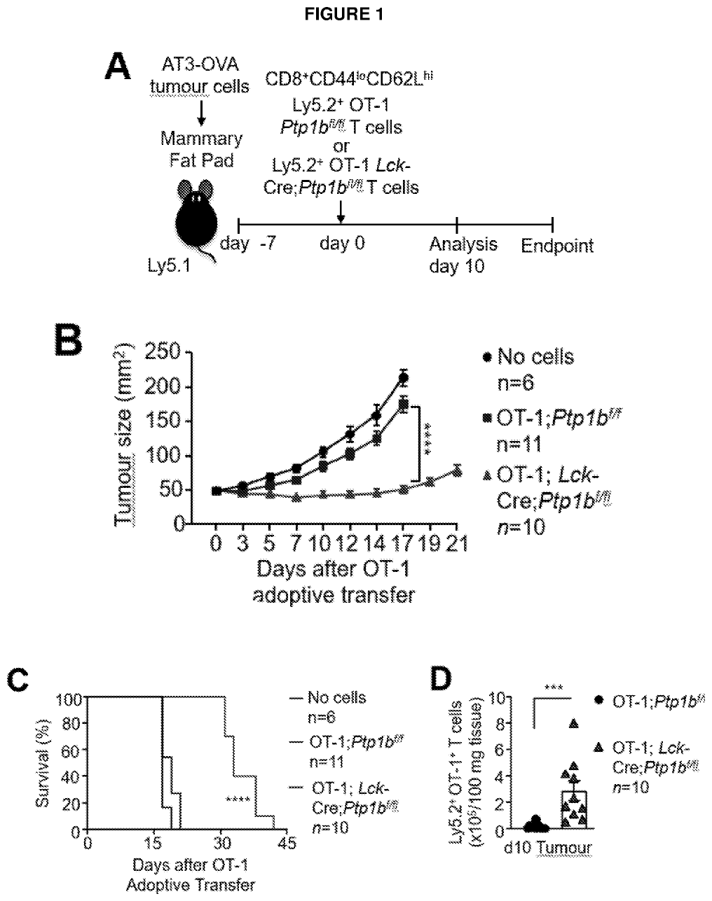

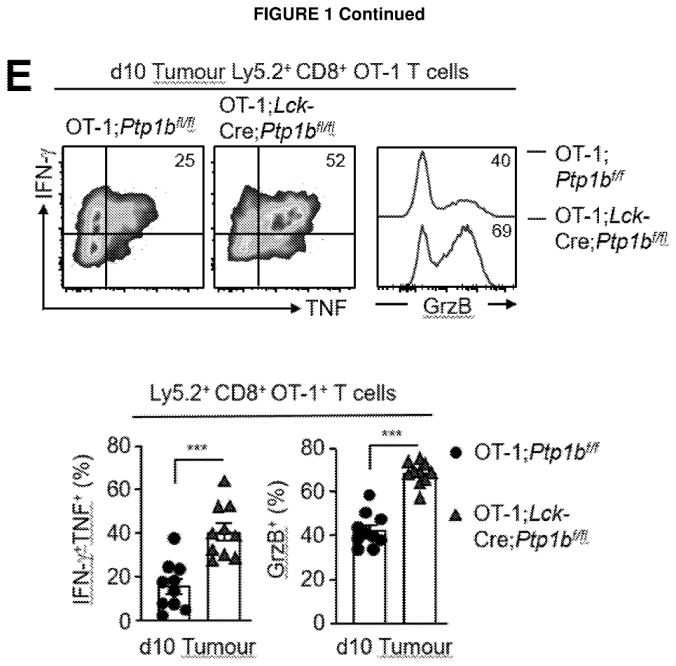

These results (shown in FIG. 1) demonstrate that adoptively transferred Ly5.2⁺;OT-1;Lck-Cre;Ptpn1^{fl/fl} T cells undergo enhanced expansion and exhibit increased survival in vivo in tumour-bearing mice. The adoptive transfer of PTP1B-deficient CD8⁺ T cells alone into tumour-bearing mice is sufficient to repress tumour growth by enhancing the number, activation and cytotoxicity of intra-tumoural CD8⁺ T cells. This prolongs the survival of mice.

### Example 2: Generation of PTP1B-Deficient CAR T Cells Exhibit Increased Antigen-Specific Activation and Cytotoxicity In Vitro

Splenocytes from Lck-Cre;Ptpnlfl/fl mice (n=4) and wild-type Ptpn1^{fl/fl} mice (n=4) were stimulated with 1 μg/ml anti-CD3 and 1 μg/ml anti-CD28 antibodies supplemented with 10 ng/ml IL-2 and 0.2 ng/ml IL-7 on d0. Cells were then transduced twice with a retrovirus encoding a second generating chimeric antibody receptor (CAR) consisting of an extracellular scFv-anti-human HER-2, a membrane proximal CD8 hinge region and the transmembrane and the cytoplasmic signalling domains of CD28 fused to the cytoplasmic region of CD3ζ (scFc-anti-HER-2-CD28-ζ) on d1 and d2. Transduced cells were then cultured with 10 ng/ml IL-2 and 0.2 ng/ml IL-7 in complete T cell medium until d7 for phenotype analysis and d8 for cytotoxic assays. The resulting CAR T cells had a mixed CD44^{hi}CD62L^{hi} central (approximately 60%) and CD44^{hi}CD62L^{lo} effector memory (approximately 40%) phenotype.
A) CAR-T cells were co-cultured with HER-2 expressing 24JK target cells versus HER-2 negative 24JK cells 4 hours prior to analysis and CD25, PD-1 and Lag-3 MFIs on CD8⁺ CAR-T cells were determined by flow cytometry.
B) CAR-T cells were co-cultured with HER-2 expressing 24JK target cells versus HER-2 negative 24JK at different ratios 4 hours prior to analysis and intracellular IFNγ in CD8⁺ CAR-T cells was determined by flow cytometry.
C) CAR-T cells were incubated with 5 μM CTV-labelled (CTV^{bright}) 24JK-HER-2 cells and 0.5 μM CTV-labelled (CTV^{dim}) 24JK sarcoma cells. Antigen-specific target cell lysis (24JK-HER-2 versus 24JK response) was assessed by monitoring for the depletion of CTV^{bright} 24JK-HER-2 cells by flow cytometry.

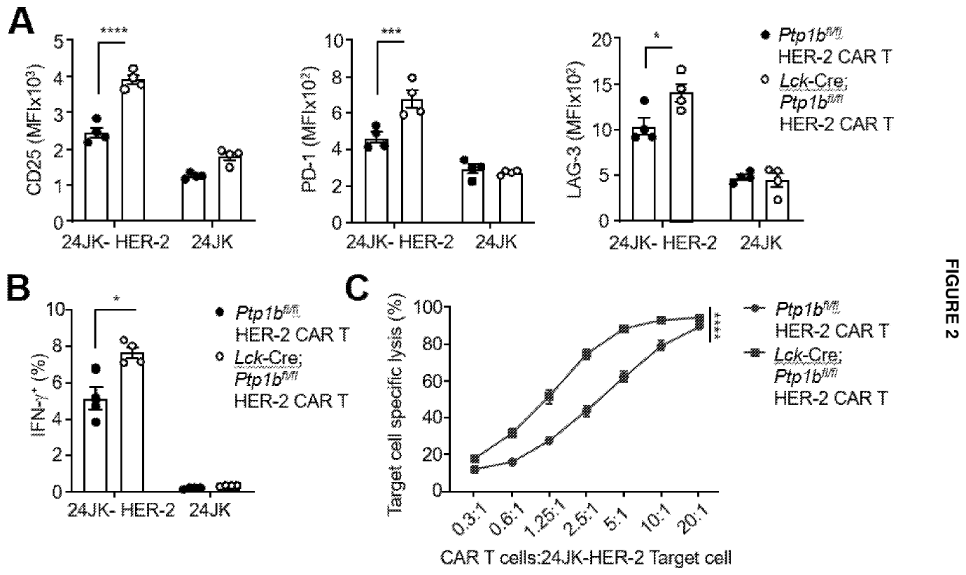

These results (FIG. 2) show that PTP1B-deficiency enhances CAR T cell activation and cytotoxicity ex vivo.

### Example 3: Generation of CAR T Cells Deficient in PTP1B and PTPN2

Ptpn2 was deleted in HER-2 CAR T cells (C57BL/6 “wild type” or Ptp1b^{-/-}) using Cas9 ribonucleoprotein (RNP)-mediated gene-editing.

Briefly, total wild type or Ptp1^{-/-} HER-2 CAR T cells generated as described in Example 2, were transfected with recombinant Cas9 (74 pmol; Alt-R S.p. Cas9 Nuclease V3, IDT) pre-complexed with short guide (sg) RNAs (600 pmol; Synthego) using the P3 Primary Cell 4D-Nucleotfector XTM Kit (Lonza Bioscence), according to the manufacturer's instructions. The sgRNAs used were:

1)
for targeting the Ptpn2 locus (Ptpn2;
5′-AAGAAGUUACAUCUUAACAC-3′; SEQ ID NO: 105)
or
2)
non-targeting sgRNAs (GCACUACCAGAGCUAACUCA;
SEQ ID NO: 106) as a control.

At day 3 post transfection cells were stained for CD8 and fixed in 200 μl Cytofix™ Fixation Buffer (BD Biosciences) for 15 min at 37° C. Cells were washed twice with D-PBS to remove excess paraformaldehyde and permeabilized in 200 μl methanol/acetone (50:50) at −20° C. overnight and then stained for intracellular PTPN2 (clone 6F3) for 30 minutes at room temperature. Secondary antibodies against mouse IgG (H+L) F(ab′)₂ fragment conjugated to AlexaFluor 647 (Molecular Probes) was used to detect PTPN2 by flow cytometry.

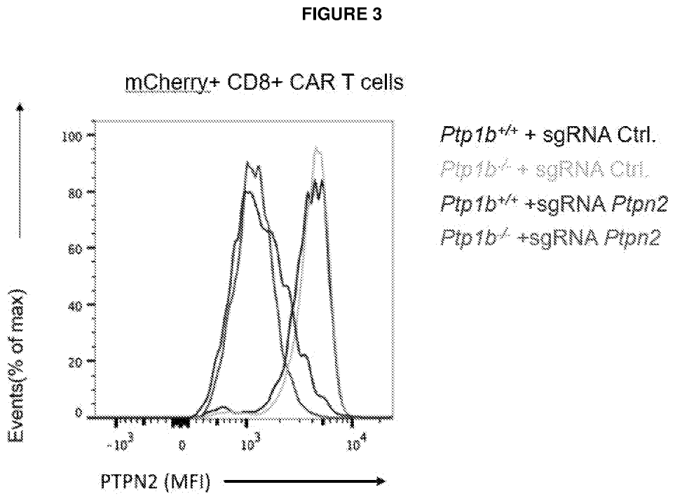

These results (shown in FIG. 3) show that CRISPR/Cas9 RNP mediates efficient PTPN2 deletion in CAR T cells.

### Example 4: Deletion of PTP1B and PTPN2 in CAR T Cells Synergistically Enhances Tumour Specific Responses In Vitro

CRISPR-RNP gene editing was used to generate HER2-specific CAR T cells deficient in both PTP1B and PTPN2 as described in Example 3.

The resulting HER2 CAR T cells were then incubated with 24JK-HER-2 versus 24JK sarcoma cells and stained for CD8, intracellular IFNγ and TNF. The proportion of CD8⁺IFNγ⁺ CAR T cells and CD8⁺TNF⁺ CAR T Cells was determined by flow cytometry. Representative results (means±SEM) from at least two independent experiments are shown. Significance was determined using 2-way ANOVA Test; ****p<0.0001.

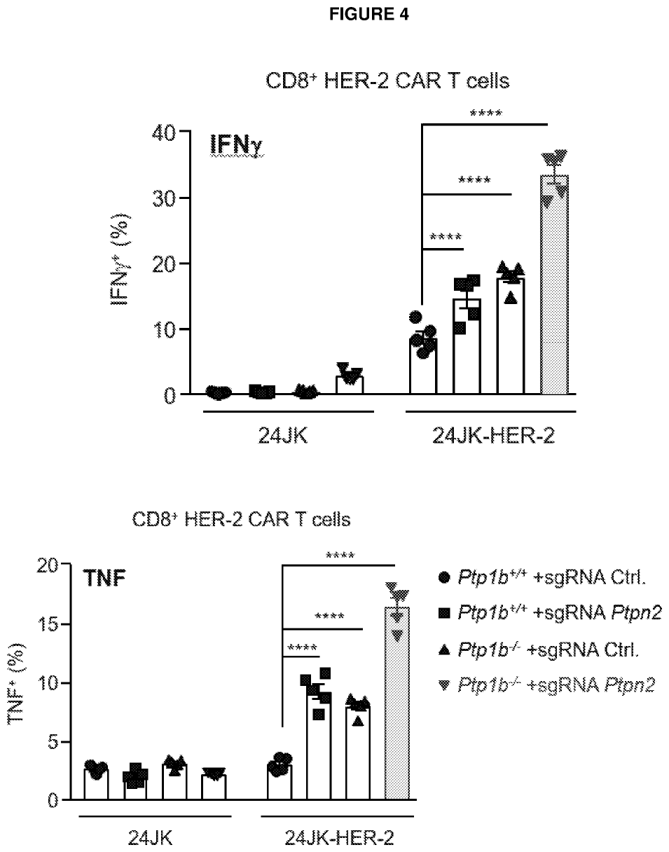

These results (shown in FIG. 4), show that combined deletion of PTPN2 and PTP1B in CAR T cells further enhances CAR T cell cytotoxicity capacity ex vivo.

### Example 5: Deletion of PTP1B and PTPN2 in CAR T Cells “Supercharqes” CAR T Cells and Enhances their Capacity to Kill Tumour Cells In Vitro

CRISPR-RNP gene editing was used to generate HER2-specific CAR T cells deficient in both PTP1B and PTPN2.

The resulting CAR T cells were incubated with 5 μM CTV-labelled (CTV^{bright}) 24JK-HER-2 cells and 0.5 μM CTV-labelled (CTV^{dim}) 24JK sarcoma cells. Antigen-specific target cell lysis (24JK-HER-2 versus 24JK response) was assessed by monitoring for the depletion of CTV^{bright} 24JK-HER-2 cells by flow cytometry.

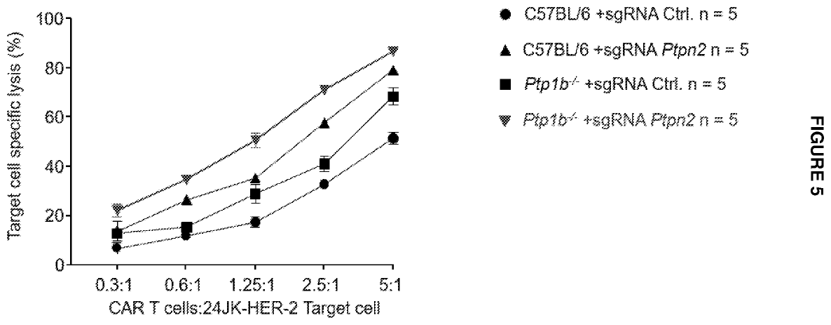

The results (FIG. 5) show that combined deletion of PTPN2 and PTP1B in CAR T cells dramatically enhances CAR T cell mediated tumour-specific cell lysis ex vivo.

### Example 6: Deletion of PTP1B and PTPN2 in CAR T Cells Enhances Tumour Responses In Vivo

HER-2-E0771 mammary tumour cells (2×10⁵) were injected into the fourth inguinal mammary fat pads of female HER-2 TG mice. Seven days after tumour injection HER-2 TG mice received total body irradiation (4 Gy) followed by the adoptive transfer of total 5×10⁶ HER-2 CAR T cells (=0.8×10⁶ viable mCherry⁺HER2 CAR⁺CD8⁺CD44^{hi}CD62L^{hi} T cells) control (Ptp1b+/+) or PTP1B-null (Ptp1b^{-/-}) HER-2 CAR T cells transfected with control (Ctrl) or Ptpn2-specific sgRNAS to delete PTPN2 by CRISPR-RNP; mice were not administered IL-2.

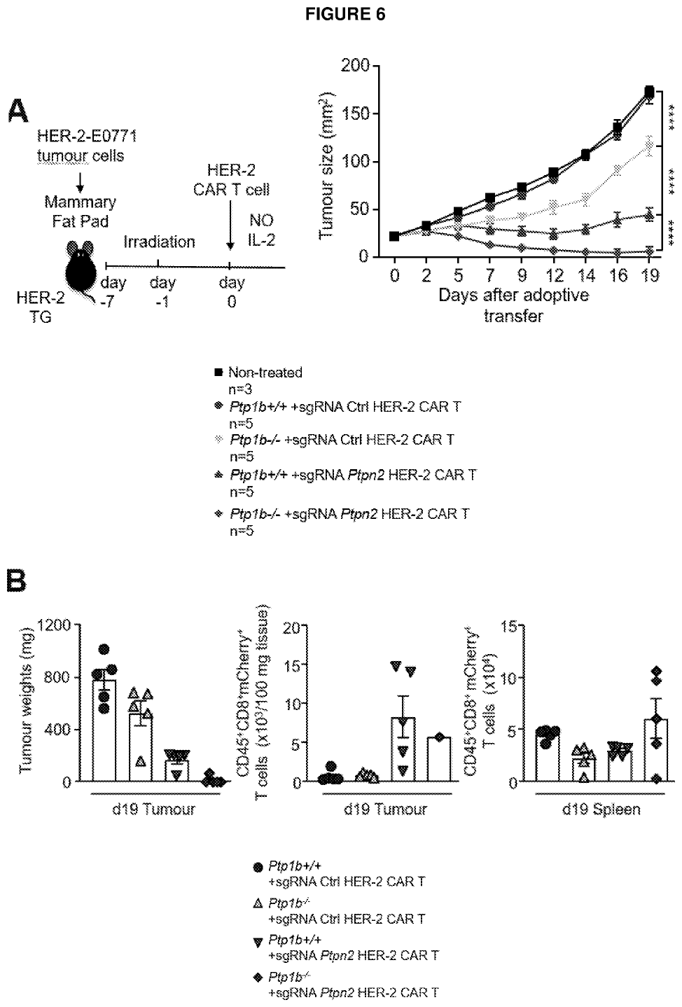

These results (FIG. 6) show that combined deletion of PTPN2 and PTP1B in CAR T cells dramatically enhances the activity of CAR T cells (even when suboptimal numbers of CAR T cells are transferred) leading to the complete eradication of HER-2 expressing tumours in vivo.

FIG. 6A) HER-2 mice were monitored for tumour growth and FIG. 6B) tumour weights and CD45⁺CD8⁺mCherry⁺ CAR T cell infiltrates in tumours and spleen determined by flow cytometry. Significance in (A) was determined using 2-way ANOVA Test; ****p<0.0001.

### Example 7: CRISPR-Cas9/RNP-Mediated PTP1B Deletion in Human T Cells Enhances TCR-Mediated Activation and Proliferation

CRISPR-RNP gene editing was used to delete PTP1B in human PBMC-derived T cells obtained from four individual donors [PBMCs stimulated with a-CD3 (OKT3) and IL-2 for 72 h] and were processed for immunoblotting, intracellular p-STAT-5, Bcl-xL or Bcl-2 (MFIs) analysis by flow cytometry, o re-stimulated with a-CD3 overnight for the analysis of CD69 (MFIs) by flow cytometry.

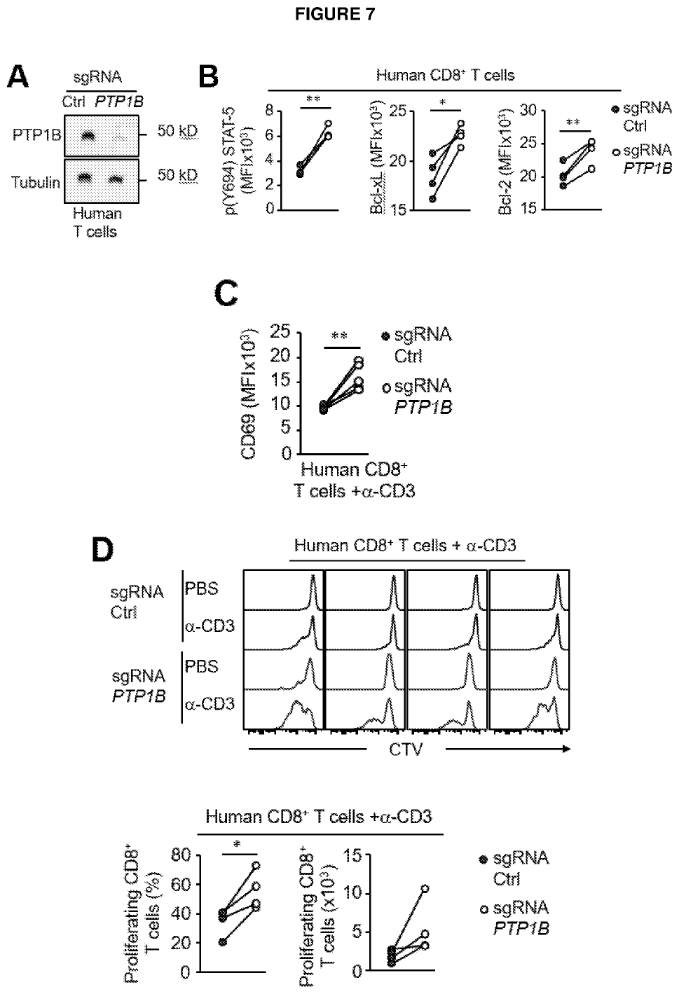

Alternatively, CTV-labelled control and PTP1B-deficient PBMC-derived human T cells were stimulated with plate-bound a-CD3 (OKT3) for 5 days and T cell proliferation (CTV dilution) assessed by flow cytometry. Representative results (means±SEM) from at least two independent experiments are shown. In (FIGS. 7B-D) significance was determined using 1-way ANOVA Test; *p<0.05, **p<0.01.

CRISPR-RNP was performed as described in Example 3. The sgRNAs used were:

1)
for targeting the PTPN1 locus (5′-UAAAAAUGGAAGAAG
CCCAA; SEQ ID NO: 107)
or
2)
non-targeting sgRNAs (GCACUACCAGAGCUAACUCA;
SEQ ID NO: 106) as a control.

The results (FIG. 7) show that that PTP1B-deficiency promotes STAT-5/Bcl-2 signalling to facilitate the TCR mediated expansion and activation of human T cells as it does in murine T cells.

### Example 8: PTP1B-Deficiency Enhances the Tumour-Specific Activity of HER2 CAR T Cells In Vivo

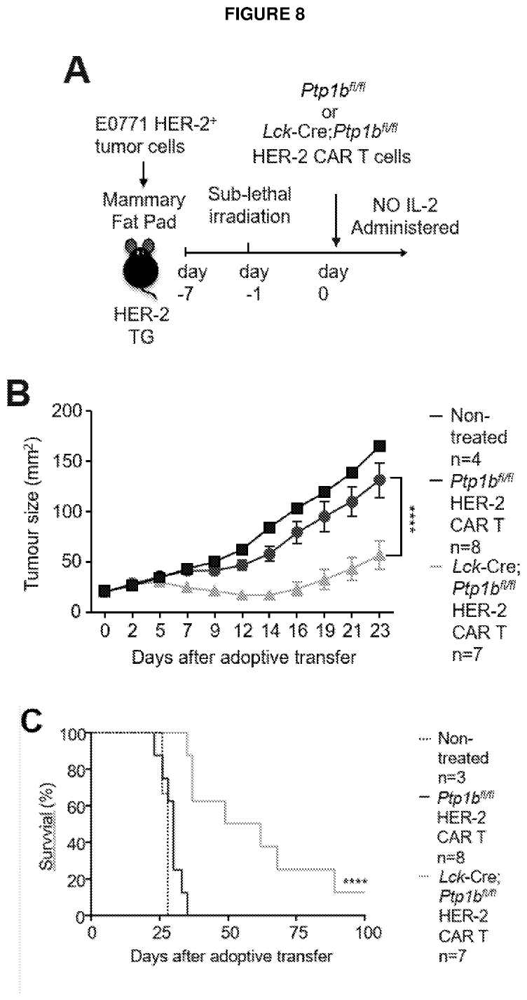

HER-2-E0771 mammary tumour cells (2×10⁵) were injected into the fourth inguinal mammary fat pads of female HER-2 TG mice. Seven days after tumour injection HER-2 TG mice received total body irradiation (4 Gy) followed by the adoptive transfer of total 20×10⁶ HER-2 CAR T cells (=6×10⁶ viable mCherry⁺HER2 CAR⁺CD8⁺CD44^{hi}CD62L^{hi} T cells) generated from Ptp1b^{fl/fl} versus Lck-Cre;Ptp1b^{fl/fl} splenocytes and monitored for tumour growth and survival. Representative results (means±SEM) from at least two independent experiments are shown. Significance in FIG. 8B was determined using 2-way ANOVA Test and in FIG. 8C using Log-rank (Mantel-Cox) test; ****p<0.0001.

These results (FIG. 8) show that PTP1B-deficiency in murine CD8+ CAR T cell promotes anti-tumour immunity and survival.

### Example 9: CRISPR-Cas9/RNP-Mediated PTP1B Deletion in Human Lewis Y (LY) CAR T Cells Enhances the Generation of Central Memory CAR T Cells and Promotes CAR T Cell Activation

CRISPR RNP was used to delete PTP1B in human PBMC-derived LY CAR T cells from four individual donors (PBMCs stimulated with OKT3 and IL-2 for 72 h and then transduced with a retrovirus encoding a CAR consisting of an extracellular scFv-anti-human LeY domain, a membrane proximal CD8 hinge region and the transmembrane and the cytoplasmic signaling domains of CD28 fused to the cytoplasmic region of CD3z. LY CAR T cells were processed for immunoblotting, or stained with fluorophore-conjugated antibodies to determine the frequency of CD8⁺LY⁺CD45RO⁺CD62L⁺ central memory CAR T cells.

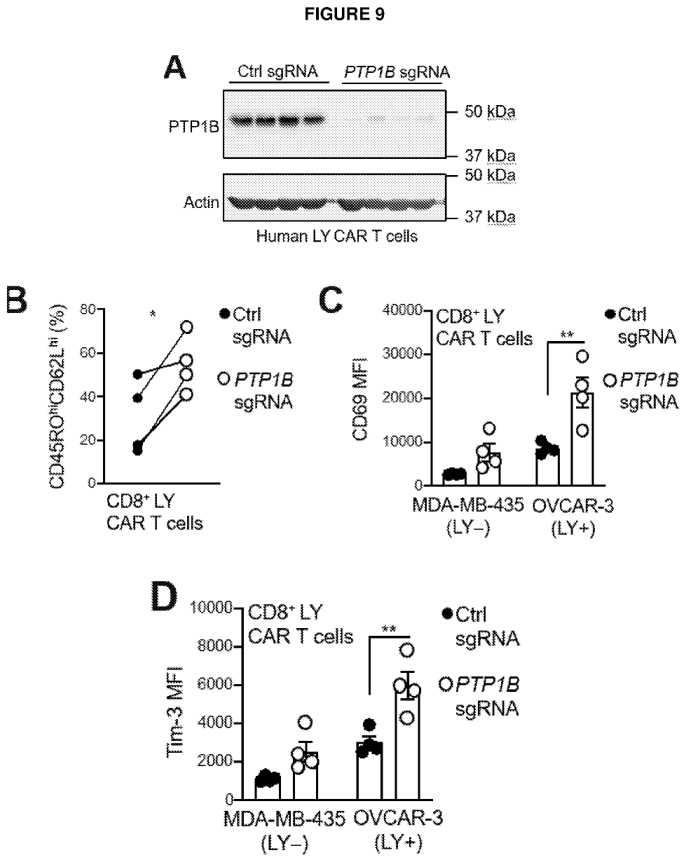

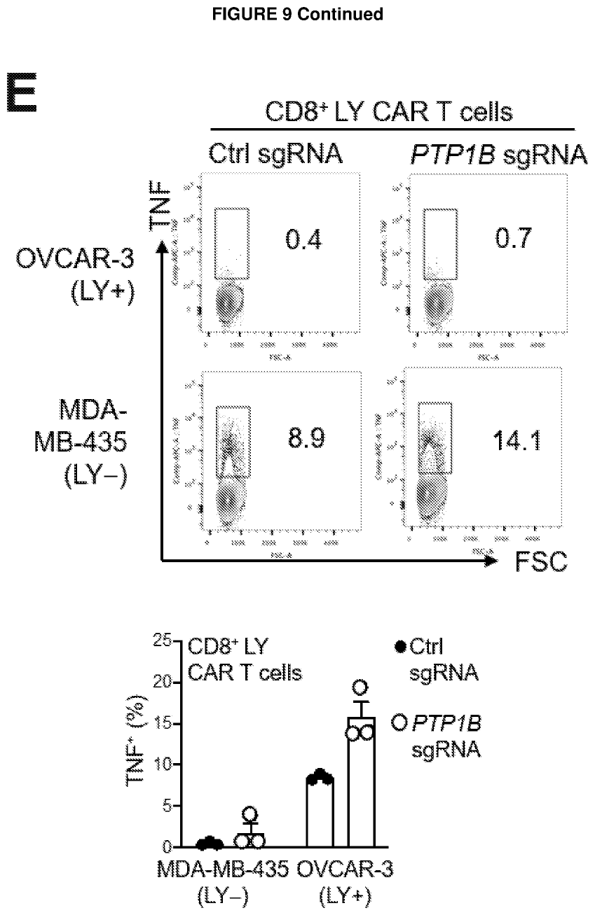

Alternatively, CD8⁺LY⁺ CAR T cells were incubated with LY-negative MDA-MB-435 cells and LY-expressing OVCAR-3 cells for the analysis of c) CD69 mean fluorescence intensity (MFI), d) Tim-3 MFI or intracellular TNF by flow cytometry. Representative results (means±SEM) from at least two independent experiments are shown. In (FIG. 9B) significance was determined using 1-way ANOVA Test, in (FIG. 9C-D) using 2-way ANOVA Test; *p<0.05, **p<0.01.

These results (FIG. 9) show that PTP1B-deficiency in human Lewis Y CAR T cells promotes the generation of long-lived, early-stage memory CAR T cells that engraft better into the host as well as the tumour antigen-specific activation of human CAR T cells.

### Example 10: PTP1B-Inhibition with MSI-1436 Enhances the Tumour-Specific Activity of HER2 CAR T Cells In Vivo

HER-2-E0771 mammary tumour cells (2×10⁵) were injected into the fourth inguinal mammary fat pads of female HER-2 TG mice. Seven days after tumour injection HER-2 TG mice received total body irradiation (4 Gy) followed by the adoptive transfer of total 20×10⁶ HER-2 CAR T cells (=6×10⁶ viable mCherry⁺HER2 CAR⁺CD8⁺CD44^{hi}CD62L^{hi} T cells) generated from Ptp1b^{fl/fl} versus Lck-Cre;Ptp1b^{fl/fl} splenocytes; mice were not administered IL-2. Mice were treated with PTP1B specific allosteric inhibitor MSI-1436 (5 mg/kg intraperitoneally) or saline on days 1, 4, 7, 10, 13, 16 and 19 post adoptive transfer and tumour growth was monitored. Representative results (means±SEM) from at least two independent experiments are shown. Significance was determined using 2-way ANOVA Test; *p<0.05, ****p<0.0001.

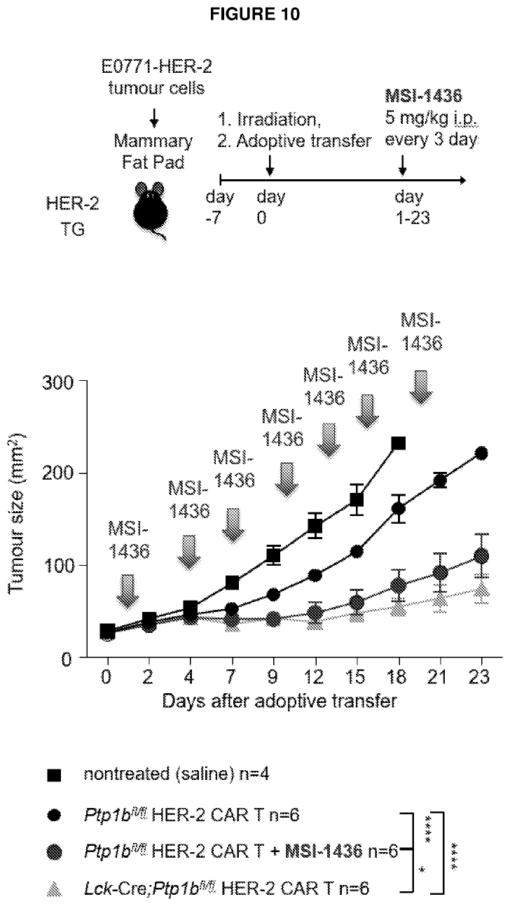

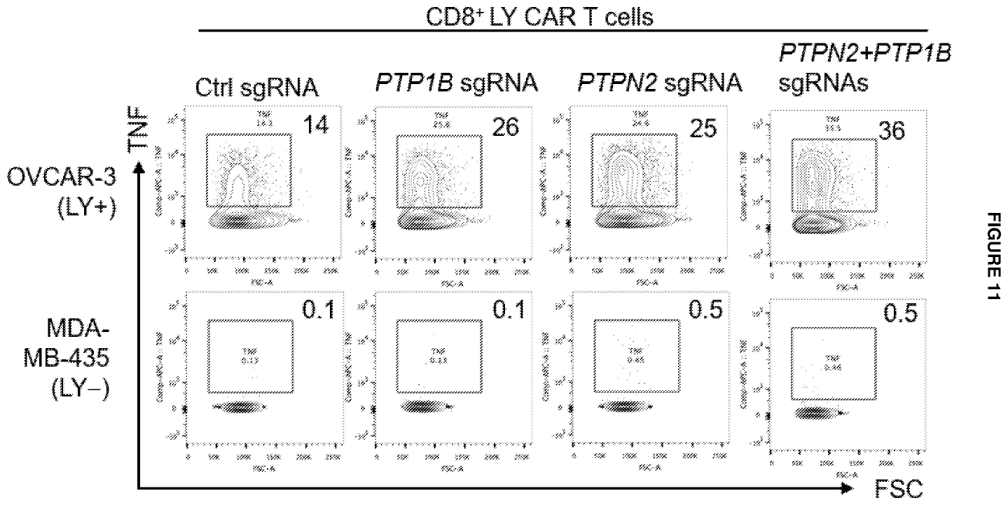

These results (FIG. 10) show that pharmacological inhibition in murine CD8⁺ HER2 CAR T cells promotes anti-tumour immunity and is as effective as the genetic deletion of PTP1B in CAR T cells.

### Example 11: Combined Deletion of PTP1B and PTPN2 in Human Lewis Y (LY) CAR T Cells Further Enhances the CAR T Cell Activation In Vitro

CRISPR RNP was used to delete PTP1B and PTPN2 in human PBMC-derived LY CAR T cells from 3 individual donors (PBMCs stimulated with OKT3 and IL-2 for 72 h and then transduced with a retrovirus encoding a CAR consisting of an extracellular scFv-anti-human LeY domain, a membrane proximal CD8 hinge region and the transmembrane and the cytoplasmic signaling domains of CD28 fused to the cytoplasmic region of CD3z. LY⁺ CAR T cells were incubated with LY-negative MDA-MB-435 cells and LY-expressing OVCAR-3 cells and intracellular TNF in CD8⁺LY⁺ CAR T cells was determined by flow cytometry. Representative results (means±SEM) from at least two independent experiments are shown.

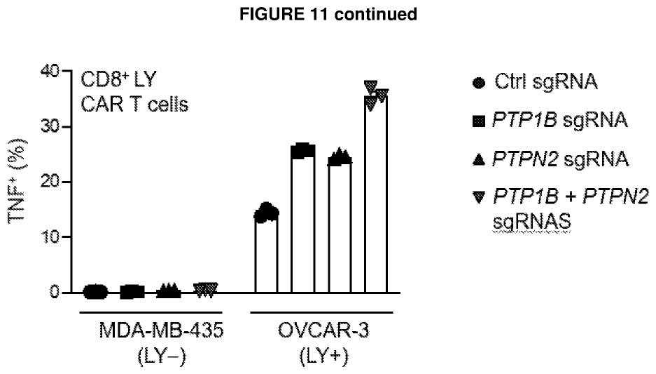

These results (FIG. 11) show that combined deletion of PTP1B and PTPN2 in human Lewis Y CAR T cells further enhances tumour antigen-specific activation.

### Example 12: Combined Inhibition of PTP1B and Deletion of PTPN2 in HER2 CAR T Cells Further Enhances the CAR T Cell Activation In Vivo

HER-2-E0771 mammary tumour cells (2×10⁵) were injected into the fourth inguinal mammary fat pads of female HER-2 TG mice. Seven days after tumour injection HER-2 TG mice received total body irradiation (4 Gy) followed by the adoptive transfer of total 5×10⁶ HER-2 CAR T cells (=0.8×10⁶ viable mCherry⁺HER-2 CAR⁺CD8⁺CD44^{hi}CD62L^{hi} T cells) control (ctrl) or Ptpn2^{-/-} HER-2 CAR CD8 T cells (PTPN2 had been deleted by CRISPR-RNP). HER-2 mice were treated with MSI-1436 (5 mg/kg intraperitoneally) or saline on days 1, 4, and 7 post adoptive transfer and tumour growth was monitored. Significance in was determined using 2-way ANOVA Test; ****p<0.0001.

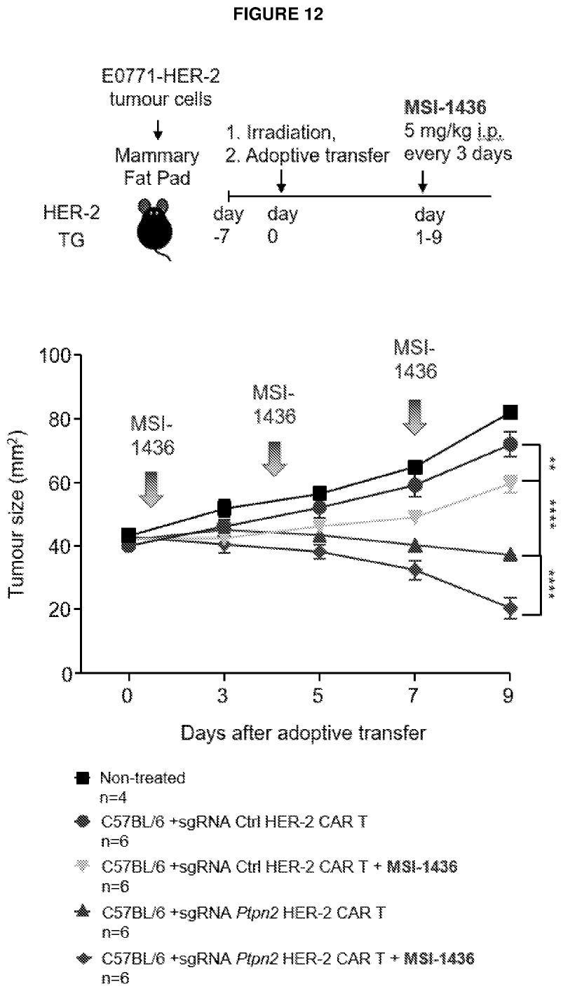

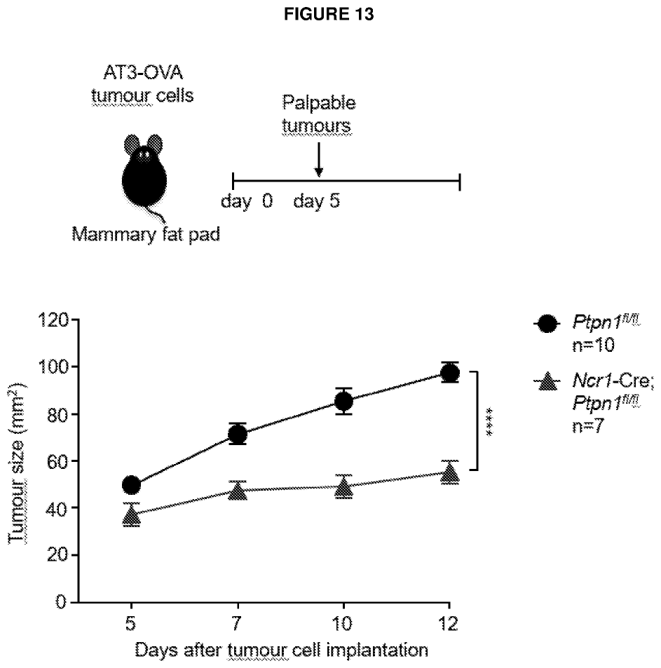

The result (FIG. 12) shows that deletion of PTPN2 and pharmaceutical inhibition of PTP1B in CAR T cells dramatically enhances the activity of CAR T cells (even when suboptimal numbers of CAR T cells are transferred) leading to the strong suppression of HER-2 expressing tumours in vivo.

### Example 13: PTP1B-Deficiency Enhances NK (Natural Killer) Cell-Mediated Anti-Tumour Immunity

AT-3-OVA mammary tumour cells (5×10⁵) were injected into the fourth inguinal mammary fat pads of female Ptpn1^{fl/fl} or NK cell specific PTP1B-deficient Ncr1-Cre;Ptpn1^{fl/fl} mice and tumour growth was monitored. Significance was determined using 2-way ANOVA Test; ****p<0.0001.

These results show that PTP1B-deficiency in NK cells is sufficient to promote anti-tumour immunity.

1. A method for producing a leukocyte that has an enhanced capacity for killing a target cell, the method comprising
contacting the leukocyte with a PTP1B inhibitor and a PTPN2 inhibitor in conditions for enabling inactivation of PTP1B and PTPN2 in the leukocyte,
thereby producing a leukocyte that has an enhanced capacity for killing a target cell.

2. The method of claim 1, wherein the leukocyte is contacted with the PTP1B inhibitor and the PTPN2 inhibitor in the absence of a T helper cell.

3. The method of claim 1 or 2, wherein the leukocyte is contacted ex vivo with the PTP1B inhibitor and the PTPN2 inhibitor.

4. A method for preparing an ex vivo population of cytotoxic leukocytes exhibiting at least one property of a cytotoxic cell, comprising culturing cytotoxic leukocytes in the presence of a PTP1B inhibitor and a PTPN2 inhibitor.

5. A method for preparing an ex vivo population of cytotoxic leukocytes exhibiting at least one property of a cytotoxic cell comprising the steps of:
culturing a cytotoxic leukocyte population from a biological sample in the presence of a PTP1B inhibitor and a PTPN2 inhibitor;
expanding the cells in culture;
thereby preparing an ex vivo population of cytotoxic leukocytes exhibiting cytotoxic properties.

6. The method of claim 5, wherein the biological sample is derived from a subject having a cancer or wherein the cytotoxic leukocytes have been conditioned or engineered to have specificity for a cancer.

7. An ex vivo method for preparing a composition comprising antigen-specific cytotoxic leukocytes, the method comprising:
providing a population of leukocytes;
co-culturing antigenic material with the leukocyte population in the presence of a PTP1B inhibitor and a PTPN2 inhibitor; and
expanding the cells in culture,
thereby preparing a composition comprising antigen-specific cytotoxic leukocytes ex vivo.

8. A method for increasing the level of cytotoxic leukocytes in a subject exhibiting an effector memory phenotype comprising the steps of:
culturing a cytotoxic leukocyte population ex vivo in the presence of a PTP1B inhibitor and a PTPN2 inhibitor;
expanding the cells in culture;
administering the cultured cells to the subject;
thereby increasing the level of cytotoxic leukocytes in a subject exhibiting an effector memory phenotype.

9. A method for forming an immune response in a subject suitable for the treatment of cancer comprising the steps of
obtaining cytotoxic leukocytes from the subject or a histocompatible donor subject (preferably a healthy donor subject);
culturing the cytotoxic leukocytes in the presence of a PTP1B inhibitor and a PTPN2 inhibitor ex vivo for a sufficient time and under conditions for to generate a population of cells exhibiting at least one cytotoxic cell property, thereby forming a population of cytotoxic leukocytes,
administering the population of cytotoxic leukocytes to the subject,
thereby producing an immune response in a subject suitable for the treatment of cancer.

10. A method of increasing CD8+ T cell mediated immunity in a subject having a disease state, preferably cancer, comprising:
contacting CD8+ T cells with a PTP1B inhibitor and a PTPN2 inhibitor ex vivo for a sufficient time and under conditions to generate a population of CD8+ T cells in which the level or activity of PTP1B and PTPN2 is depleted;
administering the population of CD8+ T cells to the subject,
thereby increasing CD8+ T cell mediated immunity in a subject.

11. A method of increasing CD8+ T cell mediated immunity in a subject having a disease state, preferably cancer, comprising:
isolating a population of the subject's CD8+ T cells;
introducing a nucleic acid molecule encoding an siRNA or shRNA directed to PTP1B into the isolated CD8+ T cells, thereby reducing the level of PTP1B in the CD8+ T cells;
contacting the CD8+ T cells with a PTPN2 inhibitor for a sufficient time and under conditions to reduce or inhibit the level or activity of PTPN2 in the CD8+ T cells;
reintroducing the CD8+ T cells into said subject,
thereby increasing the CD8+ T cell mediated immunity in a subject.

12. A method of increasing CD8+ T cell mediated immunity in a subject having a disease state, preferably cancer, comprising:
isolating a population of the subject's CD8+ T cells;
introducing a nucleic acid molecule encoding an siRNA or shRNA directed to PTPN2 into the isolated CD8+ T cells, thereby reducing the level of PTPN2 in a CD8+ T cells;
contacting the CD8+ T cells with a PTP1B inhibitor for a sufficient time and under conditions to reduce or inhibit the level or activity of PTP1B in the CD8+ T cells;
reintroducing the CD8+ T cells into said subject,
thereby increasing the CD8+ T cell mediated immunity in a subject.

13. A method of increasing CD8+ T cell mediated immunity in a subject having a disease state, preferably cancer, comprising:
isolating a population of the subject's CD8+ T cells;
introducing a Cas9 molecule complexed with a gRNA directed to PTP1B into the isolated CD8+ T cells, thereby reducing the level of PTP1B in the CD8+ T cells;
contacting the CD8+ T cells with a PTPN2 inhibitor for a sufficient time and under conditions to reduce or inhibit the level or activity of PTPN2 in the CD8+ T cells;
reintroducing the CD8+ T cells into said subject,
thereby increasing the CD8+ T cell mediated immunity in a subject.

14. A method of increasing CD8+ T cell mediated immunity in a subject having a disease state, preferably cancer, comprising:
isolating a population of the subject's CD8+ T cells;
introducing a Cas9 molecule complexed with a gRNA directed to PTPN2 into the isolated CD8+ T cells, thereby reducing the level of PTPN2 in the CD8+ T cells;
contacting the CD8+ T cells with a PTP1B inhibitor for a sufficient time and under conditions to reduce or inhibit the level or activity of PTP1B in the CD8+ T cells;
reintroducing the CD8+ T cells into said subject,
thereby increasing the CD8+ T cell mediated immunity in a subject.

15. A method of treating or promoting regression of a cancer in a subject comprising the steps of:
culturing T cells, optionally wherein the T cells are obtained from a subject, in the presence of a PTP1B inhibitor and a PTPN2 inhibitor,
administering the cultured T cells to the subject,
whereupon regression of the cancer is promoted.

16. A method of treating or promoting regression of a cancer in a subject having cancer comprising the steps of:
culturing CAR-T cells specific for a tumour antigen expressed by the cancer in the presence of a PTP1B inhibitor and a PTPN2 inhibitor,
administering the cultured CAR-T cells to the subject,
whereupon regression of the cancer is promoted.

17. A method for proliferating, enriching or expanding a composition of cells comprising a CD8+ T cell, the method comprising culturing a composition of cells in a medium, the medium comprising a PTP1B inhibitor and a PTPN2 inhibitor, wherein the PTP1B inhibitor is provided in the medium to permit contact with a CD8+ T cell during culture.

18. The method of claim 17, wherein the proliferating, enriching or expanding will result in a doubling of the number of CD8+ T cells that exhibit at least one cytotoxic T cell property.

19. The method of claim 18, wherein the expanding results in 3× or 4× number of CD8+ T cells that exhibit at least one cytotoxic T cell property, preferably at least 5×, 6×, 7×, 8×, 9× or over 10×.

20. A method of treating cancer in a subject comprising administering a population of isolated or purified CD8+ T cells effective to treat the cancer, the CD8+ T cell comprising an antigen-specific T cell receptor and wherein the CD8+ T cells have been contacted with a PTP1B inhibitor and a PTPN2 inhibitor so that the level or activity of PTP1B and PTPN2 is reduced in the cells.

21. A method for increasing the level of T cells in a subject exhibiting an effector memory phenotype comprising the steps of:
administering a PTP1B inhibitor and a PTPN2 inhibitor to the subject;
thereby increasing the level of T cells in a subject exhibiting an effector memory phenotype.

22. A method for forming an immune response in a subject suitable for the treatment of cancer comprising administering a PTP1B inhibitor and a PTPN2 inhibitor to the subject, thereby producing an immune response in a subject suitable for the treatment of cancer.

23. A method of increasing CD8+ T cell mediated immunity in a subject having a disease state comprising, administering a PTP1B inhibitor and a PTPN2 inhibitor to the subject, thereby increasing CD8+ T cell mediated immunity in a subject.

24. A method of treating cancer or promoting regression of a cancer in a subject comprising administering a PTP1B inhibitor and a PTPN2 inhibitor to the subject, thereby treating cancer in the subject, or promoting regression of the cancer.

25. The method of any one of claims 21 to 24 wherein the method further comprises the administration of CAR-T cells to the individual.

26. The method of any one of claims 21 to 25, wherein the PTP1B inhibitor and/or the PTPN2 inhibitor is administered directly to the individual.

27. The method of claim 26, wherein the inhibitor is administered systemically or by any means that allows the PTP1B inhibitor and/or PTPN2 inhibitor to enter the circulation.

28. The method of any one of claims 1 to 19, wherein the cells are purified or substantially purified prior to culture in the presence of a PTP1B inhibitor and/or PTPN2 inhibitor.

29. A population of tumour antigen-specific cytotoxic T cells for use in adoptive immunotherapy comprising an exogenous nucleic acid coding an interfering RNA for reducing the level of PTP1B in the T cells, and an exogenous nucleic acid coding an interfering RNA for reducing the level of PTPN2 in the T cells.

30. An isolated, purified or recombinant cell comprising an antigen-specific T cell receptor and an exogenous nucleic acid encoding an interfering RNA for reducing the level of PTP1B in the T cells, and an exogenous nucleic acid coding an interfering RNA for reducing the level of PTPN2 in the T cells.

31. The population of cells of claim 29 or the isolated, purified or recombinant cell of claim 30, wherein the interfering RNA is a microRNA, shRNA, siRNA or gRNA molecule that can reduce the level of PTP1B and/or PTPN2 in a cell.

32. The isolated, purified or recombinant cell of claim 30 or 31, wherein the T cell receptor (TCR) is specific for a cancer antigen and the cell is a CD8+ T cell.

33. The cell of claim 32, wherein the CD8+ T cell is a tumour infiltrating lymphocyte or a peripheral blood lymphocyte isolated from a host afflicted with cancer.

34. A composition of cytotoxic cells wherein greater than 20% of the cells have complete or partial inhibition of PTP1B and of PTPN2.

35. The composition of claim 34, wherein, the composition includes greater than 30%, 40%, 50%, 60%, 70%, 80%, 85%, 90%, 91%, 92%, 93%, 94%, 95%, 96%, 97%, 98 or 99% of cells that have complete or partial inhibition of PTP1B, or wherein preferably, all cells in the composition have complete or partial inhibition of PTP1B; and/or wherein, the composition includes greater than 30%, 40%, 50%, 60%, 70%, 80%, 85%, 90%, 91%, 92%, 93%, 94%, 95%, 96%, 97%, 98 or 99% of cells that have complete or partial inhibition of PTPN2, or wherein preferably, all cells in the composition have complete or partial inhibition of PTPN2;

36. A composition comprising a leukocyte, a PTP1B inhibitor and a PTPN2 inhibitor.

37. The composition of any one of claims 34 to 36, wherein the composition further includes a cytokine for enhancing cell killing, such as IL-2 or IFNγ.

38. The composition of any one of claims 34 to 37, wherein the cytotoxic T cell or leukocyte is selected from the group consisting of tumour infiltrating lymphocytes, peripheral blood lymphocyte, genetically engineered to express anti-tumour T cell receptors or chimeric antigen receptors (CARs), γδ T cells, enriched with mixed lymphocyte tumour cell cultures (MLTCs) or cloned using autologous antigen presenting cells and tumour derived peptides.

39. The composition of any one of claims 34 and 37, wherein the cytotoxic T cell or leukocyte is a CAR T cell, preferably a CAR T cell that is specific for a cell surface tumour antigen, more preferably wherein the CAR T cell is specific for a tumour antigen selected from Her-2, CD19, CD171, EGFR, CD22, CD123, Lewis Y, MSLN, FAP, or CD131

40. The composition of any one of claims 34 to 39, wherein the cytotoxic cells or lymphocytes are isolated from a histocompatible donor, preferably a healthy donor, or from a cancer-bearing subject.

41. Use of a PTP1B inhibitor and a PTPN2 inhibitor in the manufacture of a medicament for:
increasing the level of T cells in a subject exhibiting an effector memory phenotype;
forming an immune response in a subject suitable for the treatment of cancer;
increasing CD8+ T cell mediated immunity in a subject having a disease state;
treating cancer in a subject;
promoting regression of a cancer in a subject having cancer; or
prolonging survival of a subject having cancer.

42. A PTP1B inhibitor and a PTPN2 inhibitor or pharmaceutical composition comprising a PTP1B inhibitor and a PTPN2 inhibitor for use in:
increasing the level of T cells in a subject exhibiting an effector memory phenotype;
forming an immune response in a subject suitable for the treatment of cancer;
increasing CD8+ T cell mediated immunity in a subject having a disease state;
treating cancer in a subject;
promoting regression of a cancer in a subject having cancer; or
prolonging survival of a subject having cancer.

43. The method of any one of claims 1 to 28, the cells of any one of claims 29 to 33, the composition of any one of claims 34 to 40, the use of claim 41, or the PTP1B inhibitor and PTPN2 inhibitor for the use of claim 42, wherein:
the PTP1B inhibitor is an interfering RNA, a small molecule inhibitor, or a Cas9 molecule complexed with a gRNA directed to PTP1B that removes or modifies all or part of the Ptp1 b gene; and/or
the PTPN2 inhibitor is an interfering RNA, a small molecule inhibitor, or a Cas9 molecule complexed with a gRNA directed to PTPN2 that removes or modifies all or part of the Ptpn2 gene.

44. The method, cells, composition, or use of claim 43, wherein the small molecule inhibitor of PTP1B is claramine or trodusquemine, or derivatives thereof.

45. The method cells, composition, or use of claim 43, wherein the small molecule inhibitor of PTPN2 is ethyl-3,4-dephospatin or compound 8 as described herein, or derivatives thereof.

46. The method cells, composition, or use of claim 43, wherein the interfering RNA is siRNA or shRNA, optionally wherein the interfering RNA is provided to the cell by a lentiviral vector.

47. The method of any one of claims 1 to 28, the cells of any one of claims 29 to 33, the composition of any one of claims 34 to 40, the use of claim 41, or the PTP1B inhibitor and PTPN2 inhibitor for the use of claim 42, wherein:
the PTP1B inhibitor is a small molecule; and
the PTPN2 inhibitor is a Cas9 molecule complexed with a gRNA directed to PTP1B that removes or modifies all or part of the Ptpn2 gene.

48. The method of any one of claims 1 to 28, wherein:
the PTP1B inhibitor and PTPN2 inhibitor are not administered to the subject or wherein with the cells are not contacted with the PTP1B inhibitor and PTPN2 inhibitor at the same time.

49. A method for forming an immune response in a subject suitable for the treatment of cancer, or for increasing CD8+ T cell immunity in a subject having cancer, or for treating or promoting regression of cancer in a subject, the method comprising the steps of:
obtaining CD8+ T cells from the subject or from a histocompatible donor subject (preferably a healthy donor subject);
subjecting the CD8+ T cells to genomic editing to remove all or part of the gene encoding PTPN2, thereby reducing the expression of the gene encoding PTPN2 in the cells;
administering the population of genetically edited CD8+ T cells to the subject,
administering a PTP1B inhibitor to the subject,
thereby producing an immune response in a subject suitable for the treatment of cancer or increasing CD8+ T cell immunity in the subject or thereby promoting regression of the cancer.

50. The method of claim 49, wherein the CD8+ T cells are also genetically modified to express a Chimeric Antigen Receptor (CAR) specific for an antigen of the cancer.

51. A method treating or promoting regression of a cancer in a subject having cancer comprising the steps of:
providing a population of CAR-T cells that bind to an antigen of the cancer;
subjecting the CAR-T cells to genomic editing to remove all or part of the gene encoding PTPN2, thereby reducing the expression of the gene encoding PTPN2 in the cells;
administering the genetically edited CAR-T cells to the subject,
administering a PTP1B inhibitor to the subject,
thereby promoting regression of the cancer in the subject.

52. The method of any one of claims 49 to 51, wherein the genomic editing to remove all or part of the gene encoding PTPN2, comprises the use of a CRISPR-Cas9 or related genome editing technique.

53. The method of any one of claims 49 to 52, wherein the PTP1B inhibitor is an interfering RNA, a small molecule inhibitor, or a Cas9 molecule complexed with a gRNA directed to PTPN2 that removes or modifies all or part of the Ptp1 b gene.

54. The method of any one of claims 49 to 53, wherein the PTP1B inhibitor is a small molecule.

55. The method of claim 54, wherein the small molecule is claramine or trodusquemine, or derivatives thereof.

56. The method of any one of claims 49 to 55, wherein the PTP1B inhibitor is administered to the subject before, after or at the same time as the genetically edited cells.

57. The method of any one of claim 6, or 9 to 28, or 49 to 56, wherein the cancer is a Her-2 positive cancer, a CD19 positive cancer, a CD171 positive cancer, an EGFR-positive cancer, a CD22-positive cancer, a CD123-positive cancer, a Lewis Y positive cancer cells, or an MSLN-positive cancer, an FAP-positive cancer, or CD131-positive cancer.

58. The method of any one of claims 1 to 4, 7, 8, 10, 15-20, or 49 to 56, the cells of any one of claims 29 to 33, or the composition of any one of claims 34 to 40, wherein the cell is derived from an iPSC or ESC.


SEQUENCE LISTING (107 sequences)

SEQ ID NO: 1 - DNA, 29 bp, Artificial Sequence
  Anti-PTP1B shRNA
aattgcaccaggaagataatgactatatc

SEQ ID NO: 2 - DNA, 23 bp, Artificial Sequence
  Sense anti-PTP1B siRNA
uagguacagagacgucagudtdt

SEQ ID NO: 3 - DNA, 23 bp, Artificial Sequence
  Anti-sense anti-PTP1B siRNA
acugacgucucuguaccuadtdt

SEQ ID NO: 4 - DNA, 23 bp, Artificial Sequence
  Sense anti-PTP1B siRNA
uagguacagagacgucagudtdt

SEQ ID NO: 5 - DNA, 23 bp, Artificial Sequence
  Anti-sense anti-PTP1B siRNA
acugacgucucuguaccuadtdt

SEQ ID NO: 6 - DNA, 21 bp, Artificial Sequence
  Sense anti-PTP1B siRNA
aaatcaacggaagaagggtct

SEQ ID NO: 7 - DNA, 21 bp, Artificial Sequence
  Sense anti-PTP1B siRNA
  misc_feature (1)..(2): n is a, c, g, t or u
nnugaccauagucggauuaaa

SEQ ID NO: 8 - DNA, 25 bp, Artificial Sequence
  Sense anti-PTP1B siRNA
uugauguaguuuaauccgacuaugg

SEQ ID NO: 9 - DNA, 25 bp, Artificial Sequence
  Anti-sense anti-PTP1B siRNA
ccauagucggauuaaacuacaucaa

SEQ ID NO: 10 - DNA, 58 bp, Artificial Sequence
  PTPN1 shRNA hairpin
ccggtttgaccatagtcggattaaactcgagtttaatccgactatggtcaaatttttg

SEQ ID NO: 11 - DNA, 58 bp, Artificial Sequence
  PTPN1 shRNA hairpin
ccggctgtgatcgaaggtgccaaatctcgagatttggcaccttcgatcacagtttttg

SEQ ID NO: 12 - DNA, 58 bp, Artificial Sequence
  PTPN1 shRNA hairpin
ccggcctaacacatgcggtcactttctcgagaaagtgaccgcatgtgttaggtttttg

SEQ ID NO: 13 - DNA, 58 bp, Artificial Sequence
  PTPN1 shRNA hairpin
ccggtgcgacagctagaattggaaactcgagtttccaattctagctgtcgcatttttg

SEQ ID NO: 14 - DNA, 58 bp, Artificial Sequence; identical to SEQ ID NO: 13
  PTPN1 shRNA hairpin

SEQ ID NO: 15 - DNA, 58 bp, Artificial Sequence
  PTPN1 shRNA hairpin
ccgggaagcccaaaggagttacattctcgagaatgtaactcctttgggcttctttttg

SEQ ID NO: 16 - DNA, 58 bp, Artificial Sequence; identical to SEQ ID NO: 15
  PTPN1 shRNA hairpin

SEQ ID NO: 17 - DNA, 20 bp, Artificial Sequence
  PTPN1 gRNA target sequence
ttcgagcagatcgacaagtc

SEQ ID NO: 18 - DNA, 20 bp, Artificial Sequence
  PTPN1 gRNA target sequence
gatgtagtttaatccgacta

SEQ ID NO: 19 - DNA, 20 bp, Artificial Sequence
  PTPN1 gRNA target sequence
gagctgggcggccatttacc

SEQ ID NO: 20 - DNA, 20 bp, Artificial Sequence
  PTPN1 gRNA target sequence
tgacgtctctgtacctattt

SEQ ID NO: 21 - DNA, 20 bp, Artificial Sequence
  PTPN1 gRNA target sequence
caaaagtgaccgcatgtgtt

SEQ ID NO: 22 - DNA, 20 bp, Artificial Sequence
  PTPN1 gRNA target sequence
gtctttcagttgaccatagt

SEQ ID NO: 23 - DNA, 20 bp, Artificial Sequence
  PTPN1 gRNA target sequence
ggtaagaatgtaactccttt

SEQ ID NO: 24 - DNA, 20 bp, Artificial Sequence
  PTPN1 gRNA target sequence
gggtaagaatgtaactcctt

SEQ ID NO: 25 - DNA, 20 bp, Artificial Sequence
  PTPN1 gRNA target sequence
gatgtagtttaatccgacta

SEQ ID NO: 26 - DNA, 20 bp, Artificial Sequence
  PTPN1 gRNA target sequence
gtgtgggagcagaaaagcag

SEQ ID NO: 27 - DNA, 20 bp, Artificial Sequence
  PTPN1 gRNA target sequence
ggtgtgggagcagaaaagca

SEQ ID NO: 28 - DNA, 20 bp, Artificial Sequence
  PTPN1 gRNA target sequence
gagaaaggttcggtaagtct

SEQ ID NO: 29 - DNA, 20 bp, Artificial Sequence
  PTPN1 gRNA target sequence
gaccgcatgtgttaggcaaa

SEQ ID NO: 30 - DNA, 20 bp, Artificial Sequence
  PTPN1 gRNA target sequence
ggccctttgcctaacacatg

SEQ ID NO: 31 - DNA, 20 bp, Artificial Sequence
  PTPN1 gRNA target sequence
gtctttcagttgaccatagt

SEQ ID NO: 32 - DNA, 20 bp, Artificial Sequence
  PTPN1 gRNA target sequence
gtcacttttgggagatggtg

SEQ ID NO: 33 - DNA, 20 bp, Artificial Sequence
  PTPN1 gRNA target sequence
ggaagtcactggcttcatgt

SEQ ID NO: 34 - DNA, 20 bp, Artificial Sequence
  PTPN1 gRNA target sequence
gaagcttggccactctacat

SEQ ID NO: 35 - DNA, 20 bp, Artificial Sequence
  PTPN1 gRNA target sequence
ggaagcttggccactctaca

SEQ ID NO: 36 - DNA, 20 bp, Artificial Sequence
  PTPN1 gRNA target sequence
gctatgtgttgctgttgaac

SEQ ID NO: 37 - DNA, 20 bp, Artificial Sequence
  PTPN1 gRNA target sequence
gtgcactgcagtgcaggcat

SEQ ID NO: 38 - DNA, 20 bp, Artificial Sequence
  PTPN1 gRNA target sequence
ggtcactcagcccggagcac

SEQ ID NO: 39 - DNA, 20 bp, Artificial Sequence
  PTPN1 gRNA target sequence
gttgtggtgcactgcagtgc

SEQ ID NO: 40 - DNA, 20 bp, Artificial Sequence
  PTPN1 gRNA target sequence
ggctgagtgaccctgactct

SEQ ID NO: 41 - DNA, 20 bp, Artificial Sequence
  PTPN1 gRNA target sequence
gattcagggactccaaagtc

SEQ ID NO: 42 - DNA, 20 bp, Artificial Sequence
  PTPN1 gRNA target sequence
gactccaaagtcaggccatg

SEQ ID NO: 43 - DNA, 20 bp, Artificial Sequence
  PTPN1 gRNA target sequence
ggctgatacctgcctcttgc

SEQ ID NO: 44 - DNA, 20 bp, Artificial Sequence
  PTPN1 gRNA target sequence
gctggtaaggaggccctcgc

SEQ ID NO: 45 - DNA, 20 bp, Artificial Sequence
  PTPN1 gRNA target sequence
gtaggagaagcgcagctggt

SEQ ID NO: 46 - DNA, 20 bp, Artificial Sequence
  PTPN1 gRNA target sequence
gaaggtgccaaattcatcat

SEQ ID NO: 47 - DNA, 20 bp, Artificial Sequence
  PTPN1 gRNA target sequence
gaaatgaggaagtttcggat

SEQ ID NO: 48 - DNA, 20 bp, Artificial Sequence
  PTPN1 gRNA target sequence
ggtgaaggaagagacccagg

SEQ ID NO: 49 - DNA, 20 bp, Artificial Sequence
  PTPN1 gRNA target sequence
gttcttcccaaatcaccagt

SEQ ID NO: 50 - DNA, 20 bp, Artificial Sequence
  PTPN1 gRNA target sequence
gctgctctttcaaggatcag

SEQ ID NO: 51 - DNA, 20 bp, Artificial Sequence
  PTPN1 gRNA target sequence
ggtggggggatatgctcggg

SEQ ID NO: 52 - DNA, 20 bp, Artificial Sequence
  PTPN1 gRNA target sequence
gggtctcttccttcacccac

SEQ ID NO: 53 - DNA, 20 bp, Artificial Sequence
  PTPN1 gRNA target sequence
ggagctttcccacgaggacc

SEQ ID NO: 54 - DNA, 20 bp, Artificial Sequence
  PTPN1 gRNA target sequence
ggctccaggattcgtttggg

SEQ ID NO: 55 - DNA, 20 bp, Artificial Sequence
  PTPN1 gRNA target sequence
ggataaagactgccccatca

SEQ ID NO: 56 - DNA, 20 bp, Artificial Sequence
  PTPN1 gRNA target sequence
ggaaacataccctgtagcag

SEQ ID NO: 57 - DNA, 20 bp, Artificial Sequence
  PTPN1 gRNA target sequence
gttagaagtcgggtcgtggg

SEQ ID NO: 58 - DNA, 20 bp, Artificial Sequence
  PTPN1 gRNA target sequence
ggagccgtcactgcccgaga

SEQ ID NO: 59 - DNA, 20 bp, Artificial Sequence
  PTPN1 gRNA target sequence
gggaagtcttcgaggtgccc

SEQ ID NO: 60 - DNA, 20 bp, Artificial Sequence
  PTPN1 gRNA target sequence
ggtcaacatgtgcgtggcta

SEQ ID NO: 61 - DNA, 20 bp, Artificial Sequence
  PTPN1 gRNA target sequence
gggcagtgacggctcccctt

SEQ ID NO: 62 - DNA, 20 bp, Artificial Sequence
  PTPN1 gRNA target sequence
gtgacggctcccctttggct

SEQ ID NO: 63 - DNA, 20 bp, Artificial Sequence
  PTPN1 gRNA target sequence
gtcgtggggggaagtcttcg

SEQ ID NO: 64 - DNA, 23 bp, Artificial Sequence
  Anti-PTPN2 siRNA
aagauugacagacaccuaauauu

SEQ ID NO: 65 - DNA, 21 bp, Artificial Sequence
  Anti-PTPN2 siRNA
aagcccauaugaucacagucg

SEQ ID NO: 66 - DNA, 57 bp, Artificial Sequence
  Anti-PTPN2 shRNA hairpin
ccgggatgaccaagagatgctgtttctcgagaaacagcatctcttggtcatcttttt

SEQ ID NO: 67 - DNA, 57 bp, Artificial Sequence
  Anti-PTPN2 shRNA hairpin
ccggtgcaagatacaatggaggagactcgagtctcctccattgtatcttgcattttt

SEQ ID NO: 68 - DNA, 57 bp, Artificial Sequence
  Anti-PTPN2 shRNA hairpin
ccgggaagatgtgaagtcgtattatctcgagataatacgacttcacatcttcttttt

SEQ ID NO: 69 - DNA, 57 bp, Artificial Sequence
  Anti-PTPN2 shRNA hairpin
ccgggtgcagtagaatagacatcaactcgagttgatgtctattctactgcacttttt

SEQ ID NO: 70 - DNA, 57 bp, Artificial Sequence
  Anti-PTPN2 shRNA hairpin
ccggctcactttcattatactacctctcgagaggtagtataatgaaagtgagttttt

SEQ ID NO: 71 - DNA, 58 bp, Artificial Sequence
  Anti-PTPN2 shRNA hairpin
ccggattctcatacatggctataatctcgagattatagccatgtatgagaattttttg

SEQ ID NO: 72 - DNA, 58 bp, Artificial Sequence
  Anti-PTPN2 shRNA hairpin
ccggatatgatcacagtcgtgttaactcgagttaacacgactgtgatcatattttttg

SEQ ID NO: 73 - DNA, 58 bp, Artificial Sequence; identical to SEQ ID NO: 72
  Anti-PTPN2 shRNA hairpin

SEQ ID NO: 74 - DNA, 58 bp, Artificial Sequence
  Anti-PTPN2 shRNA hairpin
ccgggtggagaaagaatcggttaaactcgagtttaaccgattctttctccactttttg

SEQ ID NO: 75 - DNA, 58 bp, Artificial Sequence
  Anti-PTPN2 shRNA hairpin
ccggtatgatcacagtcgtgttaaactcgagtttaacacgactgtgatcatatttttg

SEQ ID NO: 76 - DNA, 57 bp, Artificial Sequence
  Anti-PTPN2 shRNA hairpin
ccgggccaagattgacagacacctactcgagtaggtgtctgtcaatcttggcttttt

SEQ ID NO: 77 - DNA, 58 bp, Artificial Sequence
  Anti-PTPN2 shRNA hairpin
ccgggtgcagtagaatagacatcaactcgagttgatgtctattctactgcactttttg

SEQ ID NO: 78 - DNA, 20 bp, Artificial Sequence
  PTPN2 gRNA target sequence
ctcttcgaactcccgctcga

SEQ ID NO: 79 - DNA, 20 bp, Artificial Sequence
  PTPN2 gRNA target sequence
agttggatactcagcgtcgc

SEQ ID NO: 80 - DNA, 20 bp, Artificial Sequence
  PTPN2 gRNA target sequence
ccatgactatcctcatagag

SEQ ID NO: 81 - DNA, 20 bp, Artificial Sequence
  PTPN2 gRNA target sequence
ccactctatgaggatagtca

SEQ ID NO: 82 - DNA, 20 bp, Artificial Sequence
  PTPN2 gRNA target sequence
ctcttctatgtcaactaaac

SEQ ID NO: 83 - DNA, 20 bp, Artificial Sequence
  PTPN2 gRNA target sequence
cagtttagttgacatagaag

SEQ ID NO: 84 - DNA, 20 bp, Artificial Sequence
  PTPN2 gRNA target sequence
ttcgaactcccgctcgatgg

SEQ ID NO: 85 - DNA, 20 bp, Artificial Sequence
  PTPN2 gRNA target sequence
ccatgactatcctcatagag

SEQ ID NO: 86 - DNA, 20 bp, Artificial Sequence
  PTPN2 gRNA target sequence
ttgacatagaagaggcacaa

SEQ ID NO: 87 - DNA, 21 bp, Artificial Sequence
  Anti-PTPN1 shRNA target
tttgaccatagtcggattaaa

SEQ ID NO: 88 - DNA, 21 bp, Artificial Sequence
  Anti-PTPN1 shRNA target
ctgtgatcgaaggtgccaaat

SEQ ID NO: 89 - DNA, 21 bp, Artificial Sequence
  Anti-PTPN1 shRNA target
cctaacacatgcggtcacttt

SEQ ID NO: 90 - DNA, 21 bp, Artificial Sequence
  Anti-PTPN1 shRNA target
tgcgacagctagaattggaaa

SEQ ID NO: 91 - DNA, 21 bp, Artificial Sequence
  Anti-PTPN1 shRNA target
gaagcccaaaggagttacatt

SEQ ID NO: 92 - DNA, 21 bp, Artificial Sequence
  Anti-PTPN1 shRNA target
gctgctctgctatatgcctta

SEQ ID NO: 93 - DNA, 21 bp, Artificial Sequence
  Anti-PTPN12 shRNA target
gatgaccaagagatgctgttt

SEQ ID NO: 94 - DNA, 21 bp, Artificial Sequence
  Anti-PTPN12 shRNA target
tgcaagatacaatggaggaga

SEQ ID NO: 95 - DNA, 21 bp, Artificial Sequence
  Anti-PTPN12 shRNA target
gaagatgtgaagtcgtattat

SEQ ID NO: 96 - DNA, 21 bp, Artificial Sequence
  Anti-PTPN12 shRNA target
gtgcagtagaatagacatcaa

SEQ ID NO: 97 - DNA, 21 bp, Artificial Sequence
  Anti-PTPN12 shRNA target
ctcactttcattatactacct

SEQ ID NO: 98 - DNA, 21 bp, Artificial Sequence
  Anti-PTPN12 shRNA target
attctcatacatggctataat

SEQ ID NO: 99 - DNA, 21 bp, Artificial Sequence
  Anti-PTPN12 shRNA target
agaagatgtgaagtcgtatta

SEQ ID NO: 100 - DNA, 21 bp, Artificial Sequence
  Anti-PTPN12 shRNA target
atatgatcacagtcgtgttaa

SEQ ID NO: 101 - DNA, 21 bp, Artificial Sequence
  Anti-PTPN12 shRNA target
gtggagaaagaatcggttaaa

SEQ ID NO: 102 - DNA, 21 bp, Artificial Sequence
  Anti-PTPN12 shRNA target
tatgatcacagtcgtgttaaa

SEQ ID NO: 103 - DNA, 21 bp, Artificial Sequence
  Anti-PTPN12 shRNA target
gccaagattgacagacaccta

SEQ ID NO: 104 - DNA, 21 bp, Artificial Sequence
  Anti-PTPN12 shRNA target
gtgcagtagaatagacatcaa

SEQ ID NO: 105 - DNA, 20 bp, Artificial Sequence
  Anti-PTPN2 sgRNA
aagaaguuacaucuuaacac

SEQ ID NO: 106 - DNA, 20 bp, Artificial Sequence
  Control non-targeting sgRNA
gcacuaccagagcuaacuca

SEQ ID NO: 107 - DNA, 20 bp, Artificial Sequence
  Anti-PTPN1 sgRNA
uaaaaauggaagaagcccaa